(19) United States
(12) Patent Application Publication
(10) Pub. No.: US 2023/0114269 A1
(43) Pub. Date: Apr. 13, 2023

(54) TECHNIQUES FOR MEASURING BLOOD OXYGEN LEVELS

(71) Applicant: Oura Health Oy, Oulu (FI)

(72) Inventors: Olli Petteri Heikkinen, Oulu (FI); Roosa Annikki Wederhorn, Espoo (FI); Xi Zhang, Daly City, CA (US); Mikko Pellervo Tuohimaa, Kempele (FI); Tero Juhani Vallius, Oulu (FI); Jussi Petteri Järvelä, Kempele (FI); Markku Koskela, Oulu (FI); Juha-Pekka Syrjälä, Oulu (FI); Teemu Juahni Haverinen, Oulu (FI); Kaisa Helena Tarvainen, Oulu (FI); Tuomas Viljami Kenttä, Liminka (FI)

(21) Appl. No.: 17/964,271

(22) Filed: Oct. 12, 2022

Related U.S. Application Data
(60) Provisional application No. 63/255,362, filed on Oct. 13, 2021.

(51) Int. Cl. A61B 5/1455; A61B 5/00; A61B 5/0205

(52) U.S. Cl. CPC A61B 5/14551; A61B 5/7289; A61B 5/742; A61B 5/02055; A61B 5/746; A61B 5/6802

(57) ABSTRACT

Methods, systems, and devices for blood oxygen measurement are described. A method may include receiving, via a wearable device, a first photoplethysmogram (PPG) signal for a user acquired during a time interval using a first set of PPG sensors, and receiving a second PPG signal for the user acquired during the time interval using a second set of PPG sensors that are different from the first set of PPG sensors. The method may include comparing the first PPG signal and the second PPG signal, and determining one or more blood oxygen saturation metrics for the user during the time interval based on the comparison of the first PPG signal and the second PPG signal. The method may include causing a graphical user interface (GUI) to display an indication of the one or more blood oxygen saturation metrics.

### CROSS REFERENCE

The present Application for Patent claims the benefit of U.S. Provisional Patent Application No. 63/255,362 by HEIKKINEN et al., entitled “TECHNIQUES FOR MEASURING BLOOD OXYGEN LEVELS,” filed Oct. 13, 2021, assigned to the assignee hereof, and expressly incorporated by reference herein.

### FIELD OF TECHNOLOGY

The following relates to wearable devices and data processing, including techniques for measuring blood oxygen levels.

### BACKGROUND

Some wearable devices may be configured to collect physiological data from users, including temperature data, heart rate data, and the like. However, poor contact between a user's skin and one or more sensors of a wearable device may result in inaccurate measurements, particularly in the context of blood oxygen saturation measurements.

### BRIEF DESCRIPTION OF THE DRAWINGS

FIG. 1 illustrates an example of a system that supports techniques for measuring blood oxygen levels in accordance with aspects of the present disclosure.

FIG. 2 illustrates an example of a system that supports techniques for measuring blood oxygen levels in accordance with aspects of the present disclosure.

FIG. 3 illustrates an example of a wearable device diagram that supports techniques for measuring blood oxygen levels in accordance with aspects of the present disclosure.

FIG. 4 illustrates an example of a process flow that supports techniques for measuring blood oxygen levels in accordance with aspects of the present disclosure.

FIG. 5 illustrates an example of a timing diagram that supports techniques for measuring blood oxygen levels in accordance with aspects of the present disclosure.

FIG. 6 illustrates an example of a graphical user interface (GUI) that supports techniques for measuring blood oxygen levels in accordance with aspects of the present disclosure.

FIG. 7 illustrates an example of a wearable device diagram that supports techniques for measuring blood oxygen levels in accordance with aspects of the present disclosure.

FIG. 8 illustrates an example of a wearable device diagram that supports techniques for measuring blood oxygen levels in accordance with aspects of the present disclosure.

FIG. 9 illustrates an example of a frequency diagram that supports techniques for measuring blood oxygen levels in accordance with aspects of the present disclosure.

FIG. 10 shows a block diagram of an apparatus that supports techniques for measuring blood oxygen levels in accordance with aspects of the present disclosure.

FIG. 11 shows a block diagram of a wearable application that supports techniques for measuring blood oxygen levels in accordance with aspects of the present disclosure.

FIG. 12 shows a diagram of a system including a device that supports techniques for measuring blood oxygen levels in accordance with aspects of the present disclosure.

FIGS. 13 and 14 show flowcharts illustrating methods that support techniques for measuring blood oxygen levels in accordance with aspects of the present disclosure.

### DETAILED DESCRIPTION

Some wearable devices may be configured to collect physiological data from users, including temperature data, heart rate data, photoplethysmogram (PPG) signals, motion data, and the like. In order to efficiently and accurately track physiological data, a wearable device may be configured to collect data continuously while the user wears the device. Some wearable devices may be configured to measure blood oxygen saturation levels for a user based on acquired physiological data. However, conventional wearable devices have been unable to efficiently, accurately, and reliably perform blood oxygen saturation measurements for a variety of reasons.

For example, if a wearable device is worn on a wrist of a user, the one or more sensors of the wearable device, such as one or more light-emitting diodes (LEDs), may create new optical interfaces between the skin of the user (e.g., and the arteries within the tissue) and the sensors. The new optical interfaces may behave differently as compared to cases where there is good skin contact between the skin of the user and the sensors. In such cases, the new optical interfaces may change a critical angle due to reflections, reduce perfusion index due to internal stray light, cause variations in distribution of light, and the like. The variation in optical interface and wavelength may cause inaccurate readings from the sensors, which may result in inaccurate blood oxygen measurements. In some cases, the wearable device may adjust a power of the sensors, such as increasing the brightness of an LED, to account for the variation in readings, which may increase power consumption at the wearable device. Taken together, these issues with wearable devices may result in inaccurate physiological data readings, which may lead to a distorted picture of the user's overall health (e.g., distorted blood oxygen measurements), as well as increased power consumption and decreased battery life.

Accordingly, techniques described herein are directed to systems and methods for measuring blood oxygen saturation for a user. More specifically, aspects of the present disclosure are directed to techniques for using two different sets of PPG sensors of a wearable ring device which are configured to determine one or more blood oxygen saturation metrics. By using two (or more) different sets of PPG sensors and determining one or more blood oxygen saturation metrics, techniques described herein may lead to more accurate blood oxygen saturation measurements, and may decrease a power consumption at the wearable device, which may lead to longer battery life.

As described herein, a wearable ring device may include a first light source, a second light source, a first photodetector (PD), and a second PD. The first set of PPG sensors may include the first light source, the first PD, and the second PD. The second set of PPG sensors may include the second light source, the first PD, and the second PD. The wearable ring device may further include four channels that may direct light from one of the light sources, such as a LED, to one of the PDs. PPG signals may be acquired along each of the respective channels. For example, the wearable ring device may receive a first PPG signal using a first channel and a second PPG signal using a second channel. The first channel may include a channel between the first light source and the first PD, and the second channel may include a channel between the first light source and the second PD. The wearable device may compare the first PPG signal with the second PPG signal. As such, by comparing the first PPG signal with the second PPG signal, techniques described herein may be used to determine one or more blood oxygen saturation metrics for the user, and cause a graphical user interface (GUI) to display an indication of the one or more blood oxygen saturation metrics.

A wearable ring device of the present disclosure may utilize any number of channels for acquiring PPG signals used to determine blood oxygen saturation metrics for a user. For example, a wearable ring device may also receive a third PPG signal using a third channel and a fourth PPG signal using a fourth channel. The third channel may include a channel between the second light source and the first PD, and the fourth channel may include a channel between the second light source and the second PD. In such cases, the wearable ring device may be configured to compare PPG signals received via the respective channels in order to determine more accurate and reliable blood oxygen saturation metrics for the user.

Aspects of the disclosure are initially described in the context of systems supporting physiological data collection from users via wearable devices. Additional aspects of the disclosure are described in the context of wearable user device diagrams, an example GUI, and timing diagrams. Aspects of the disclosure are further illustrated by and described with reference to apparatus diagrams, system diagrams, and flowcharts that relate to techniques for measuring blood oxygen levels.

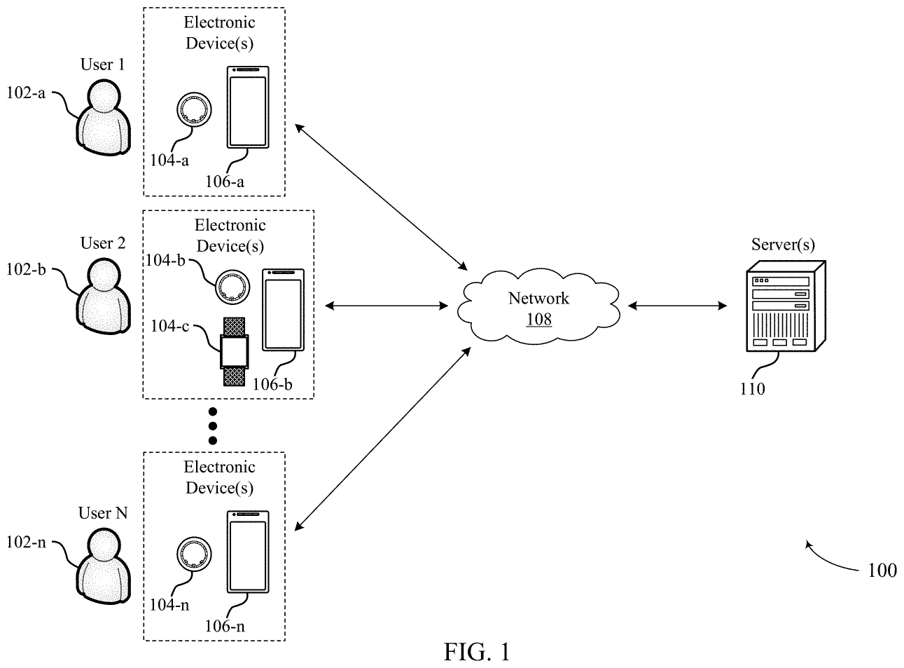

FIG. 1 illustrates an example of a system 100 that supports techniques for measuring blood oxygen levels in accordance with aspects of the present disclosure. The system 100 includes a plurality of electronic devices (e.g., wearable devices 104, user devices 106) which may be worn and/or operated by one or more users 102. The system 100 further includes a network 108 and one or more servers 110.

The electronic devices may include any electronic devices known in the art, including wearable devices 104 (e.g., wearable ring devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, which may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a wearable ring device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earwearable ring devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b , where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

In some implementations, the rings 104 (e.g., wearable devices 104) of the system 100 may be configured to collect physiological data from the respective users 102 based on arterial blood flow within the user's finger. In particular, a ring 104 may utilize one or more LEDs (e.g., red LEDs, green LEDs) which emit light on the palm-side of a user's finger to collect physiological data based on arterial blood flow within the user's finger. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices which utilize LEDs which are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time during which a user 102 is asleep, and classify periods of time during which the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time during which the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, which repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models which are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g. in a hypothetical culture with 12 day “weeks”, 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 100 may support techniques for measuring blood oxygen levels (e.g., blood oxygen saturation levels). The system 100 may receive, via a wearable device 104, a first PPG signal for a user acquired during a time interval using a first set of PPG sensors. The system 100 may receive a second PPG signal for the user acquired during the time interval using a second set of PPG sensors. The second set of PPG sensors may be different from the first set of PPG sensors. For example, the first set of PPG sensors may include at least one red LED, and the second set of PPG sensors may include at least one infrared LED. In some cases, the first set of PPG sensors or the second set of PPG sensors may include at least one green LED. In such cases, the first set of PPG sensors may include at least one red LED, and the second set of PPG sensors may include at least one green LED or the first set of PPG sensors may include at least one green LED, and the second set of PPG sensors may include at least one infrared LED.

In some implementations, the system 100 may compare the first PPG signal and the second PPG signal. The system 100 may determine one or more blood oxygen saturation metrics for the user during the time interval based on the comparison of the first PPG signal and the second PPG signal. In such cases, the system 100 may cause a GUI (e.g., GUI of a user device 106) to display an indication of the one or more blood oxygen saturation metrics.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

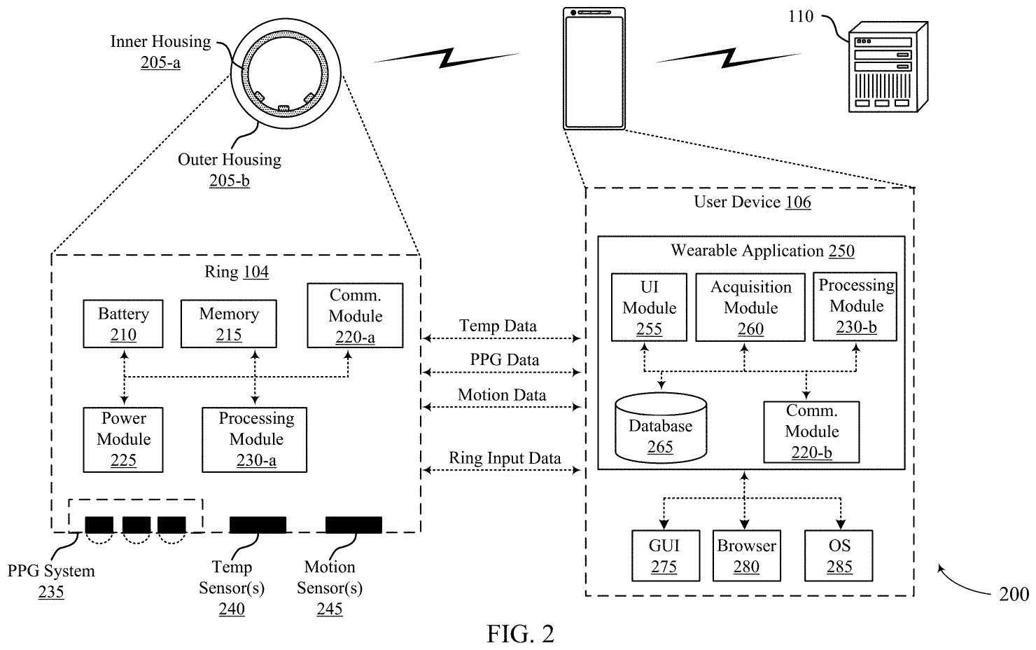

FIG. 2 illustrates an example of a system 200 that supports techniques for measuring blood oxygen levels in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels, and the like.

System 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, PPG data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205, which may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components which are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using a clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, which may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG LEDs. In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a , 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or which supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during 104 charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time.

For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 in which the optical receiver(s) receive transmitted light that is reflected through the region of the user's finger. In some implementations, the PPG system 235 may be configured as a transmissive PPG system 235 in which the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include LEDs. The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform, which may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BM1160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 200 may support techniques for measuring oxygen saturation for a user. For example, the system 200 may use multiple channels (e.g., including at least two channels that transmit red light and two channels that transmit infrared light) for collecting PPG signals. The system 200 may receive multiple PPG signals using different sets of sensors of a wearable ring device. The different sets of sensors may include at least a red LED and an infrared LED. The system 200 may compare the PPG signals and determine blood oxygen saturation metrics for the user based on the comparison. The blood oxygen saturation metrics may be displayed to the user (e.g., via GUI 275).

For example, as noted previously herein, the ring 104 of the system 200 may be worn by a user to collect physiological data from the user, including temperature, heart rate, motion, PPG signals, and the like. The ring 104 of the system 200 may collect the physiological data from the user based on arterial blood flow. The physiological data may be collected continuously. In some implementations, the processing module 230-a may sample the user's temperature continuously throughout the day and night. Sampling at a sufficient rate (e.g., one sample per minute) throughout the day may provide sufficient temperature data for analysis described herein. In some implementations, the ring 104 may continuously acquire temperature data, heart rate data, PPG data, and motion data (e.g., at a sampling rate).

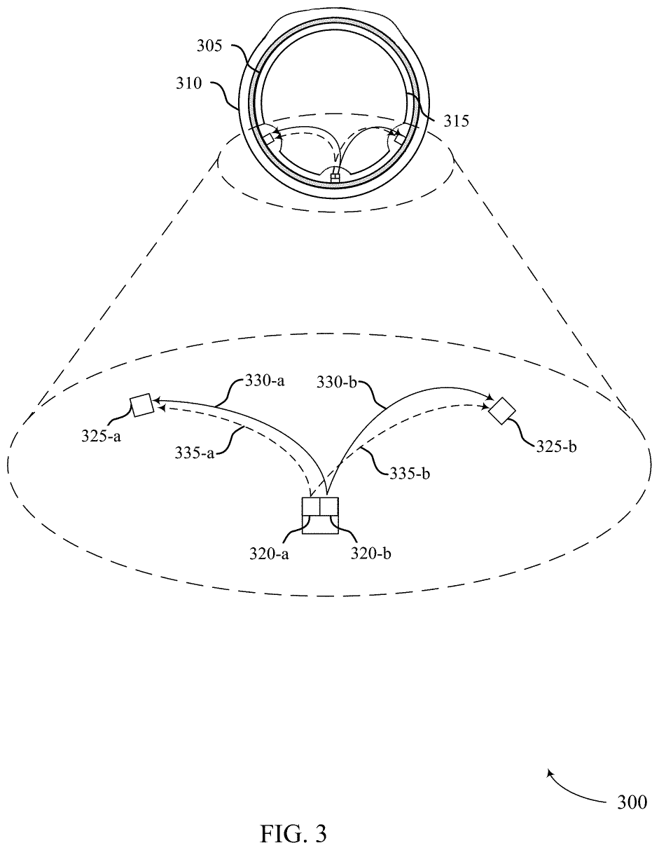

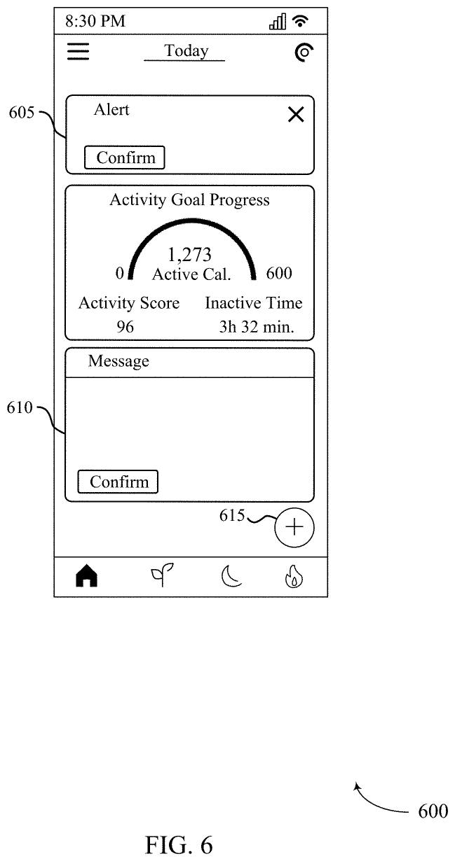

Data collected by the ring 104 may be used to determine blood oxygen saturation metrics for the user. Measuring blood oxygen saturation for the user is further shown and described with reference to FIG. 3. The user device 106 may display an alert or message at a GUI 275 of the user device 106, where the alert or message may indicate the one or more blood oxygen saturation metrics, may alert the user of the one or more blood oxygen saturation metrics exceeding a threshold, or the like. Messaging and alerts associated with blood oxygen saturation metrics are described in further detail with respect to FIG. 6.

FIG. 3 illustrates an example of a wearable device diagram 300 that supports techniques for measuring blood oxygen levels in accordance with aspects of the present disclosure. The wearable device diagram 300 may implement, or be implemented by, aspects of the system 100, system 200, or both. For example, wearable device diagram 300 may illustrate examples of wearable devices 104 as described with reference to FIGS. 1 and 2. Although the wearable devices are illustrated as rings in FIG. 3, aspects and components of the wearable devices illustrated in FIG. 3 may be implemented in any type of wearable device (e.g., a watch, a bracelet, a necklace, and the like).

In some examples, the wearable device may include an inner housing 305 and an outer housing 310, which may be examples of an inner housing 205 and outer housing 205 as described with reference to FIG. 2. One or more sensors may be embedded in the inner housing 305, such as one or more LEDs 320 for collecting physiological measurements. In some cases, an outer opaque shell may be molded over an inner structure of the wearable device. Further, the wearable device in wearable device diagram 300 may include an electronic substrate, such as a printed wiring board (PWB) or PCB. The PCB may have both flexible and rigid sections.

One or more sensors may be embedded in the electronic substrate. For example, the electronic substrate may include one or more LEDs 320 and PDs 325. The wearable device may include LED 320-a, which may emit light 335 (e.g., light 335-a, 335-b) received by PD 325-a and/or PD 325-b. In this regard, the LED 320-a may support two optical channels for physiological data measurements: a first optical channel between the LED 320-a and the PD 325-a and a second optical channel between the LED 320-a and the PD 325-b. The wearable device may include any number of LEDs, PDs, and respective optical channels for physiological data measurements. In some cases, LED 320-a may be a red LED, which may emit light 335 that is scattered and absorbed by skin 315 of a user of the wearable device diagram 300 (e.g., reflective and/or transmissive measurements). In such cases, the system 200 may measure the same light 335 via two different PDs (e.g., PD 325-a and PD 325-b) which may increase the diversity and reliability of the measurement.

The first set of PPG sensors may include a first light source (e.g., LED 320-a), PD 325-a, and PD 325-b. The first optical channel may include a channel between LED 320-a and PD 325-a where LED 320-a may emit light 335-a. The second optical channel may include a channel between LED 320-a and PD 325-b where LED 320-a may emit light 335-b. In such cases, the wearable device diagram 300 may include a 2:1 relationship between the PDs 325 and the LED 320-a.

The wearable device may include LED 320-b, which may emit light 330 received by PD 325-a and/or PD 325-b. In this regard, the LED 320-b may support two optical channels for physiological data measurements: a first optical channel between the LED 320-b and the PD 325-a and a second optical channel between the LED 320-b and the PD 325-b. In some cases, LED 320-b may be an infrared LED, which may emit light 330 that is scattered and absorbed by skin 315 of a user of the wearable device diagram 300 (e.g., reflective and/or transmissive measurements). In such cases, the system 200 may measure the same light 330 via two different PDs (e.g., PD 325-a and PD 325-b) which may increase the diversity and reliability of the measurement in addition to measuring blood oxygen measurements using two different light sources (e.g., LED 320-a and LED 320-b).

The second set of PPG sensors may include a second light source (e.g., LED 320-b), PD 325-a, and PD 325-b. In this regard, the wearable device diagram 300 illustrates four separate optical channels: two optical channels between the LED 320-a and the PDs 325-a, 325-b, and two optical channels between the LED 320-b and the PDs 325-a, 325-b. As such, the wearable device diagram 300 may include a 2:1 relationship between the PDs 325 and the LEDs 320. In some aspects, separate PPG signals may be acquired along each of the respective optical channels. Moreover, in some implementations, the LED 320-a may be configured to generate light 335 with a different wavelength as compared to the light 330 generated by the LED 320-b, which may further improve a diversity of measurements (e.g., PPG signals) acquired within the wearable device diagram 300.

The LED 320-a, which may be a red LED, may emit light 335, such that light 335-a may be guided along first channel to PD 325-a and light 335-b may be guided along second channel to PD 325-b. Similarly, LED 320-b, which may be an infrared LED, may emit light 330, such that light 330-a may be guided along a third channel to PD 325-a and light 330-b may be guided along a fourth channel to PD 325-b . The first set of PPG sensors (e.g., including LED 320-a, PD 325-a, and PD 325-b) may be configured to acquire a first PPG signal using light of a first wavelength (e.g., red light). The second set of PPG sensors (e.g., including LED 320-b, PD 325-a, and PD 325-b) may be configured to acquire a second PPG signal using light of a second wavelength (e.g., infrared light). The LED 320-a which may be an example of a red LED may emit light of 740-760 nm wavelength. In some implementations, the LED 320-b may be an example of an infrared LED. In some examples, the LED 320-a, LED 320-b, or both, may include laser diodes.

In some cases, PD 325-a, PD 325-b, or both may detect light emitted from one or more LEDs 320, such as PD 325-a and PD 325-b used for physiological measurements. In such cases, at least one PD 325 may be included within both the first set of PPG sensors (including LED 320-a) and the second set of PPG sensors (including LED 320-b). For example, the PD 325 may be used for both red and infrared PPG measurements. In some other cases, PD 325-a, PD 325-b, or both may be specific to LED 320-a or LED 320-b (e.g., only receive light transmitted by a corresponding LED 320).

In some cases, the wearable device may include an additional LED that may emit light. The additional LED may be a red LED, an infrared LED, a green LED, a blue LED, or a combination thereof. The light may be scattered and absorbed by the skin 315 of the user, and measured via the PDs 325-a and/or PD 325-b. As noted previously herein, each of the LEDs 320-a and 320-b may support multiple optical channels via the respective PDs 325-a, 325-b. The PD 325-a and PD 325-b may be configured to measure light from the respective LEDs 320 that is reflected by the skin and/or transmitted through the skin (e.g., reflective and/or transmissive measurements). In such cases, a first PPG signal (e.g., generated via measurement of light 335-a and light 335-b), the second PPG signal (e.g., generated via measurement of light 330-a and light 330-b), or both may be based on light that is transmitted through and/or reflected by tissue of the user. For example, the PPG signals may be based on a combination of transmissive and reflective light.

In some cases, each of the LED 320-a, PD 325-a, LED 320-b, and PD 325-b may be positioned at different radial positions relative to an axis of the wearable device and along an inner circumference of the wearable device. In some examples, the LED 320-a and the LED 320-b may be positioned at the same radial position relative to an axis of the wearable device and along the inner circumference of the wearable device. For example, the LED 320-a and LED 320-b may be adjacent to each other along the inner circumference of the wearable device. In some examples, the PD 325-a may be positioned at a radial position opposite of the PD 325-b. The PD 325-a may be positioned radially closer to the LED 320-a in order to make the radial distance between the PD 325-a and the LED 320-a shorter (e.g., as compared to the radial distance between the PD 325-a and the LED 320-b), and the PD 325-b may be positioned radially closer to the LED 320-b in order to make the radial distance between the PD 325-b and the LED 320-b shorter (e.g., as compared to the radial distance between the PD 325-b and the LED 320-a). In some cases, the positions of the PD 325-a and PD 325-b may be switched.

In some cases, the inner housing 305 may include a dome structure over the one or more LEDs 320, one or more PDs 325, or both. For example, the wearable device may include dome structures over LED 320-a, LED 320-b, PD 325-a, and PD 325-b to improve contact with the skin 315. In some other cases, there may be a window for the LED 320 to emit light 330 or light 335. An optical interface may form between the inner housing 305 and the domes or the windows (e.g., with a refractive index of ˜1.57) and the top layer of skin 315 (e.g., with a refractive index of ˜1.55). The wearable device may use the light propagation from the LEDs 320 to the PDs 325 through tissue for physiological measurements, such as PPG and SpO2 measurements. That is, the wearable device may use light 335 from LED 320-a, which may include red wavelengths, to measure SpO2 or PPG and light 330 from LED 320-b, which may include infrared wavelengths, to measure SpO2 or PPG. Light 330 may penetrate skin 315 to a different depth than light 335 due to the varying wavelengths.

In some examples, there may be three different types of interfaces between the LEDs 320 and the skin 315 for the wearable device in addition to the optical paths between the LEDs 320 and the PDs 325 used for physiological measurements. For example, LED 320-a and LED 320-b may be embedded in inner housing 305 (e.g., in an optically clear epoxy material) and may emit light, such as light 335 and 330, respectively, coupled out of the LED optics through an inner housing 305 to skin 315 interface. In some cases, LED 320-a and LED 320-b may be under a dome (e.g., made of epoxy, metal, or a combination thereof) and may emit light 335 and 330, respectively, coupled out of LED optics through the inner housing 305 to skin 315 interface. LEDs 320-a and 320-b may be flush with the surface of the inner housing 305, such that the skin may make direct contact with LED 320-a and LED 320-b . Further, light propagating inside the skin 315 (e.g., finger tissue) may be coupled to the PD optics through skin 315 to inner housing 305 interface.

In some implementations, a single PD 325 may be used together with multiple LEDs 320 to save cost and space. In some examples, LEDs 320-a and 320-b or both may be colored LEDs that may be used for performing physiological measurements. By measuring the signals (e.g., at PDs 325), it may be possible to use LED 320 and PD 325 pairs that have sufficient optical paths during rapid motion and reduce battery consumption.

In some cases, if a red/green/blue (RGB) LED is used, the wearable device may be able to perform spectral analysis based on acquired physiological data. That is, multiple wavelengths of light may be used for performing spectral analysis procedures, which may be used for guiding PPG wavelength selection. For example, referring to wearable device diagram 300, multiple wavelengths of light (e.g., light 330 and light 335) may be directed through the channels, where the different wavelengths of light 330 and light 335 may be coupled out of the channels differently based on a material in contact with the channels that exhibits spectral differences in refractive index or absorptance. In other words, different quantities/proportions of the respective wavelengths of light 330 and light 335 may escape the optical channels based on optical properties of a material in contact with the respective channels. Materials that may affect the optical behavior of the respective channels may include sweat, dirt, water, other liquids, and the like. In some examples, spectral analysis procedures performed by the wearable device may enable the wearable device to alert the user of one or more medical conditions.

The wearable device may be an example of a wearable ring device. For example, the PPG signals acquired along the respective channels illustrated in the wearable device diagram 300 may be acquired as a combination of the transmissive and reflective light transmitted through the skin 315 of the finger. In such cases, the system 200 may measure blood oxygen measurements at different locations of the finger by transmitting or reflecting the light through a portion of the finger (e.g., widest portion of the finger) where the smaller arteries (e.g., arterioles) and capillaries are located. Comparatively, other portions of the body (e.g., top of the wrist) may not include arterioles and may include bone and other bodily material that may interfere with physiological measurements collected by some wearable devices. As such, some other wearable devices, such as wearable devices worn around the wrist, may be unable to acquire physiological data based on blood flow within arterioles, which may result in inferior physiological measurements as compared to measurements acquired using a wearable ring device, thereby decreasing an efficiency and reliability of acquired physiological measurements.

In some cases, the system 200 may calibrate the measurements for different ring sizes and for different LED currents. The LED 320 properties may change as higher currents or different currents may be used. For example, a smaller ring size may include smaller LED currents as compared to a larger ring size. If a larger LED current were to be used with a smaller ring size, PDs 325 may be saturated such that the PD 325 may be unable to detect the light 330 or light 335. Using a smaller LED current may decrease the power consumption and increase the battery life of the wearable ring device.

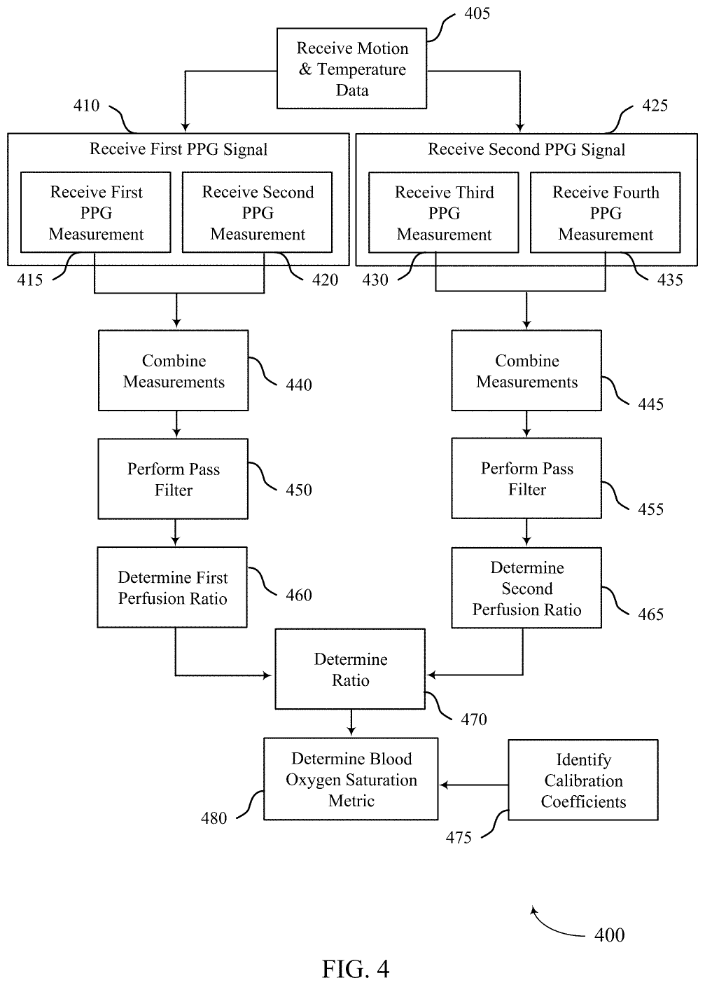

FIG. 4 illustrates an example of a process flow 400 that supports techniques for measuring blood oxygen levels in accordance with aspects of the present disclosure. The process flow 400 may be implemented by the system 200 including at least a server 110, a user device 106, a wearable device 104, or some combination of components from these devices. Alternative examples of the following may be implemented, where some steps are performed in a different order than described or not performed at all. In some cases, steps may include additional features not mentioned below, or further steps may be added.

At 405, the system 200 may receive motion and temperature data. For example, the system 200 may receive physiological data associated with the user via the wearable device. The physiological data may include at least motion data, or temperature data, or both. In such cases, the system 200 may utilize motion data and temperature data as triggers for performing a blood oxygen measurement. In other words, the system 200 may evaluate motion data, temperature data, or both, to determine time intervals that may result in high quality and accurate blood oxygen measurements. In some cases, the motion data may satisfy a threshold motion metric if the motion data is less than or equal to the threshold motion metric. The temperature data may satisfy the threshold temperature metric if the temperature is greater than or equal to the threshold temperature metric. In such cases, the trigger condition to perform a blood oxygen measurement may be if the temperature data exceeds the threshold temperature metric and if the motion data is below the threshold motion metric. For example, the system 200 may initiate a blood oxygen measurement in response to determining that the user experiences little to no motion (e.g., the motion is below a threshold) and the temperature is high (e.g., above a threshold). In such cases, the system 200 may determine an infrared perfusion with increased accuracy.

The system 200 may identify a baseline temperature associated with the user based on receiving the physiological data. In some examples, the baseline temperature may include a nighttime temperature baseline. In such cases, the temperature data may satisfy the nighttime temperature baseline (e.g., threshold temperature metric) if the temperature is greater than or equal to the nighttime temperature baseline. The received temperature data and motion data may be saved to a local memory storage.

The system 200 may receive physiological data associated with the user via the wearable device where the physiological data includes at least heart rate data or PPG signal feature data, or both. In some cases, the system 200 may determine that the heart rate satisfies a threshold heart rate metric. The system 200 may determine that the PPG signal feature data satisfies a threshold metric. In such cases, the system 200 may utilize heart rate data and other features of the PPG signal such as pulse wave amplitude attenuation to trigger performing a blood oxygen measurement.

In some examples, the system 200 may implement automatic gain control in direct response to receiving the motion data and the temperature data. Automatic gain control may monitor and adjust a brightness of the light, sensitivity of the measurement, the rate of light, a voltage or current supplied to PPG sensors (e.g., LEDs 320), or a combination thereof. The motion data and the temperature data may be received during the night (e.g., when a user is asleep). In such cases, the blood oxygen measurement may be performed with increased accuracy and precision by performing the blood oxygen measurement when the user is in an optimal position (e.g., at rest) and experiences increased body temperature. In some cases, the system 200 may conserve power associated with the wearable device (e.g., ring) by receiving the motion data and temperature data overnight and performing the blood oxygen measurement in direct response to receiving the motion data and temperature data.

At 410, the system 200 may receive a first PPG signal. For example, the system 200 may receive, via a wearable device, a first PPG signal for a user acquired during a time interval using a first set of PPG sensors. For example, the first PPG signal may be acquired using the first LED 320-a and one or more PDs 325. In some cases, receiving the first PPG signal may be based on the motion data satisfying a threshold motion metric and the temperature data satisfying a threshold temperature metric. In some examples, the system 200 may receive the first PPG signal based on the heart rate satisfying a threshold heart rate metric and the PPG signal feature data satisfying a threshold metric. The received first PPG signal may be saved to a local memory storage.

At 415, the system 200 may receive a first PPG measurement of the first PPG signal. For example, the system 200 may receive a first PPG measurement utilizing a first channel between a first subset of the first set of PPG sensors. For instance, the system 200 may acquire a first PPG measurement using the first LED 320-a and the first PD 325-a. The system 200 may receive the first PPG signal that may include (or is used to generate) the first PPG measurement. In such cases, the first PPG signal may be based on the first PPG measurement. For example, the system 200 may measure the first PPG measurement at a first PD using red light at 50 Hz. The received second PPG signal may be saved to a local memory storage.

At 420, the system 200 may receive a second PPG measurement. For example, the system 200 may receive a second PPG measurement utilizing a second channel between a second subset of the first set of PPG sensors. For instance, the system 200 may acquire a second PPG measurement using the first LED 320-a and the second PD 325-b. The system 200 may receive the first PPG signal that may include (or is used to generate) the second PPG measurement. In such cases, the first PPG signal may be based on the second PPG measurement. For example, the system 200 may measure the second PPG measurement at a second PD using red light at 50 Hz. In such cases, the first PPG signal may include red light and be measured using two different channels between the same red light source and two different PDs.

At 425, the system 200 may receive a second PPG signal. For example, the system 200 may receive, via a wearable device, a second PPG signal for a user acquired during a time interval using a second set of PPG sensors. For example, the second PPG signal may be acquired using the second LED 320-b and one or more PDs 325. In some cases, receiving the second PPG signal may be based on the motion data satisfying a threshold motion metric and the temperature data satisfying a threshold temperature metric. In some examples, the system 200 may receive the second PPG signal based on the heart rate satisfying a threshold heart rate metric and the PPG signal feature data satisfying a threshold metric.

In some cases, receiving the first PPG signal and the second PPG signal may include selectively controlling an activation state of the first light source (e.g., red light source) and the second light source (e.g., infrared light source) such that the first light source and the second light source are simultaneously in an active activation state. For example, the system 200 may fire (e.g., activate) the red light source (e.g., LED 320-a) and the infrared light source (e.g., LED 320-b) simultaneously.

In additional or alternative cases, receiving the first PPG signal and the second PPG signal may include sequentially controlling an activation state of the first light source (e.g., red light source) and the second light source (e.g., infrared light source) such that the first light source is in an active activation state when the second light source is in an inactive activation state, and vice versa. For example, the firing rate of the infrared light source (e.g., LED 320-b) may be offset from the firing rate of the red light source (e.g., LED 320-a) such that the infrared light source fires when the red light source is inactive (e.g., not firing), and vice versa. The system 200 may fire (e.g., activate) the red light source prior to activating the infrared light source, or the system 200 may fire (e.g., activate) the infrared light source prior to activating the red light source. The system 200 may fire (e.g., activate) the red light source and the infrared light source sequentially.

The system 200 may determine a breathing rate for the user based on the motion data, the first PPG signal, the second PPG signal, or a combination thereof. For example, the system 200 may use a combination of PPG signals, accelerometer measurements, and/or gyroscope measurements of the wearable device to determine (e.g., sense) the breathing rate of the user and use the breathing rate for correcting the blood oxygen measurements.

At 430, the system 200 may receive a third PPG measurement. For example, the system 200 may receive a third PPG measurement utilizing a third channel between a first subset of the second set of PPG sensors. For instance, the system 200 may acquire a third PPG measurement using the second LED 320-b and the first PD 325-a. The system 200 may receive the second PPG signal that may include the third PPG measurement. In such cases, the second PPG signal may be based on the third PPG measurement. For example, the system 200 may measure the third PPG measurement at the first PD using infrared light at 50 Hz.

At 435, the system 200 may receive a fourth PPG measurement. For example, the system 200 may receive a fourth PPG measurement utilizing a fourth channel between a second subset of the second set of PPG sensors. For instance, the system 200 may acquire a third PPG measurement using the second LED 320-b and the second PD 325-b. The system 200 may receive the second PPG signal that may include the fourth PPG measurement. In such cases, the second PPG signal may be based on the fourth PPG measurement. For example, the system 200 may measure the fourth PPG measurement at the second PD using infrared light at 50 Hz. In such cases, the second PPG signal may include infrared light and be measured using two different channels between the same infrared light source and two different PDs.

In general, the system 200 may acquire any number of PPG signals using any combination of PPG sensors in order to perform blood oxygen measurements (e.g., blood oxygen saturation measurements) described herein. For example, the system 200 may receive a third PPG signal for the user acquired during the time interval using a third set of PPG sensors that are different from the first set of PPG sensors and the second set of PPG sensors. Receiving the third PPG signal may include receiving a fifth PPG measurement utilizing a fifth channel between a first subset of the third set of PPG sensors and receiving a sixth PPG measurement utilizing a sixth channel between a second subset of the third set of PPG sensors. In such cases, the third PPG signal may be based on the fifth PPG measurement and the sixth PPG measurement. The first set of PPG sensors, the second set of PPG sensors, and the third set of PPG sensors may use three different wavelengths.

At 440, the system 200 may combine the measurements. For example, the system 200 may combine the first PPG measurement and the second PPG measurement to generate a first combined PPG signal/measurement. In some cases, the system 200 may combine the first PPG measurement and the second PPG measurement using one or more mathematical operations such as an averaging operation, a weighted averaging operation, or both. The first combined PPG signal/measurement may be based on two different PPG measurements (e.g., red channels) in which the two different PPG measurements from more than one infrared channel may be combined. The first PPG measurement and second PPG measurement may be averaged by averaging the pulse (e.g., AC amplitude) of each PPG measurement. Additionally, in some aspects, the system 200 may determine that one of the PPG measurements exhibits poor signal quality or strength. In such cases, the system 200 may drop, or otherwise ignore, the PPG measurement with the low signal quality/strength.

At 445, the system 200 may combine the measurements. For example, the system 200 may combine the third PPG measurement and the fourth PPG measurement to generate a second combined PPG signal/measurement. In some cases, the system 200 may combine the third PPG measurement and the fourth PPG measurement using one or more mathematical operations such as an averaging operation, a weighted averaging operation, or both. The second combined PPG signal/measurement may be based on two different PPG measurements (e.g., infrared channels) in which the two different PPG measurements from more than one red channel may be combined. The third PPG measurement and fourth PPG measurement may be averaged by averaging the pulse (e.g., AC amplitude) of each PPG measurement. Additionally, in some aspects, the system 200 may determine that one of the PPG measurements exhibits poor signal quality or strength. In such cases, the system 200 may drop, or otherwise ignore, the PPG measurement with the low signal quality/strength.

In some cases, the combined PPG signals (e.g., first combined PPG signal and second combined PPG signal) may include quality markers to indicate a quality (e.g., resolution, accuracy, precision, etc.) of the combined PPG signals. The quality marker may be calculated for each PPG waveform. For example, the system 200 may perform a moving kurtosis calculation of the combined PPG signals, a shape estimation of the Lissajous pattern of the PPG signal associated with the red channel and the infrared differential absorption (dA) plot, and the like.

For the purposes of the present disclosure, the term “kurtosis” may be used to refer to the fourth standardized moment, which may be defined according to Equation 1 below:

$$Kurt [ X ] = E [ {( \frac{X - μ}{σ} )}^{4} ] = \frac{E [ {( X - μ )}^{4} ]}{{( E [ {( X - μ )}^{2} ] )}^{2}} = \frac{{μ}_{4}}{{σ}^{4}} \qquad ( 1 )$$

where μ₄ is the fourth central moment and a is the standard deviation. Using Equation 1, the system 200 may be configured to calculate differences of subsequent samples (e.g., subsequent measurements), where the numerator=diff(ppg), and calculate the average of a sample and a subsequent sample (e.g., average of sequential PPG measurements), where the denominator=(ppg(1:end−1))+ppg(2:end))/2. The system 200 may then divide the numerator and the denominator values to obtain the dA. In particular, the system 200 may calculate dA for each of the respective channels (e.g., red channel, IR channel), then plot the dA metrics for the respective channels against one another to generate dA absorption plots. In some cases, the system 200 may identify characteristics of dA absorption plots (e.g., slope, R values) to determine characteristics (e.g., relative quality) of determined PPG measurements and/or blood oxygen metrics.

At 450, the system 200 may perform a pass filter. In response to averaging the first PPG measurement and the second PPG measurement, the system 200 may perform a low pass filter at 9 Hz followed by performing a high pass filter at 0.5 Hz. In some cases, the system 200 may inverse (e.g., invert) the PPG measurement after taking the average of the PPG measurements. In such cases, the system 200 may perform a low pass filter after inverting the PPG measurement.

At 455, the system 200 may perform a pass filter. In response to averaging the third PPG measurement and the fourth PPG measurement, the system 200 may perform a low pass filter followed by performing a high pass filter. In some cases, the system 200 may invert the PPG measurement after taking the average of the PPG measurements. In such cases, the system 200 may perform a low pass filter after inverting the PPG measurement.

At 460, the system 200 may determine a first perfusion ratio. For example, the system 200 may determine a first perfusion index (e.g., perfusion ratio) between a first amplitude and a first baseline amplitude level of the first PPG signal. The perfusion ratio may be equal to amplitude divided by direct current (DC). If the system 200 determines that the first perfusion ratio is greater than some threshold (e.g., 0.15%), the system 200 may determine a ratio.

At 465, the system 200 may determine a second perfusion ratio. For example, the system 200 may determine a second perfusion index (e.g., perfusion ratio) between a second amplitude and a second baseline amplitude level of the second PPG signal. The perfusion ratio may be equal to amplitude divided by DC. If the system 200 determines that the second perfusion ratio is greater than 0.15%, the system 200 may determine a ratio.

At 470, the system 200 may determine a ratio between the first combined PPG signal and the second combined PPG signal. In such cases, the system 200 may determine a ratio between the first perfusion index and the second perfusion index (e.g., a ratio of ratios). In this regard, the system 200 may compare the first combined PPG signal and the second combined PPG signal, and may generate a blood oxygen saturation signal.

At 475, the system 200 may identify calibration coefficients. For example, the system 200 may identify one or more calibration coefficients for blood oxygen saturation calculations. The calibration coefficients may include a first calibration coefficient, a second calibration coefficient, and a third calibration coefficient. In some aspects, calibration coefficients may be used along with the blood oxygen saturation signal (e.g., plugged into the blood oxygen saturation signal) in order to determine blood oxygen saturation metrics.

At 480, the system 200 may determine a blood oxygen saturation metric. For example, the system 200 may determine one or more blood oxygen saturation metrics for the user during the time interval based on the comparison of the first combined PPG signal and the second combined PPG signal. In some cases, the one or more blood oxygen saturation metrics may be determined based on the blood oxygen saturation signal and the one or more calibration coefficients, the ratio of the first combined PPG signal and the second combined PPG signal, or both. For example, the system 200 may determine the blood oxygen saturation metric by comparing the combined PPG signals from the different channels (e.g., red channel, infrared channel) and utilizes calibration coefficients to determine the blood oxygen saturation metric for the user.

Additionally, or alternatively, the system 200 (e.g., a wearable ring device of the system 200) may determine a variation (e.g., variance, variability) of the one or more blood oxygen saturation metrics. That is, the system 200 may determine an average rate of change (e.g., absolute average) between subsequent blood oxygen saturation metrics of the one or more blood oxygen saturation metrics over a time interval. Additionally, or alternatively, the system 200 (e.g., a wearable ring device of the system 200) may determine an average blood oxygen saturation metric over a first time interval and compare it to an average blood oxygen saturation metric over a second time interval to determine a variation in average blood oxygen saturation metrics from the first time interval to the second time interval. Accordingly, the variation of the one or more blood oxygen saturation metrics may be based on the average rate of change, the variation in average blood oxygen saturation metrics between time intervals, or both.

In some examples, system 200 may compare the determined variation to a threshold. The determined variation exceeding a threshold may indicate a sleep disturbance of the user. Conversely, the determined variance falling below the (e.g., failing to exceed the threshold) threshold may indicate a stable blood oxygen saturation of the user.

In some implementations, the system 200 may filter out outliers (e.g., including outlier PPG signals). The determined blood oxygen saturation metrics for the user may be saved to local memory storage. In some cases, the system 200 may generate a blood oxygen saturation signal based on the comparison of the first PPG signal and the second PPG signal. The system 200 may update the one or more blood oxygen saturation metrics for the user based on determining the breathing rate for the user. In such cases, the system 200 may use a combination of PPG signals based heart rate, accelerometer, and/or gyroscope of the wearable device to sense the breathing rate of the user and use the breathing rate of the user to correlate (e.g., correct) the blood oxygen saturation metrics (e.g., correlate when breathing disturbances result in drops in blood oxygen saturation metrics).

In some implementations, the system 200 may modify the first PPG signal and the second PPG signal based on the temperature data. In such cases, determining the one or more blood oxygen saturation metrics may be based on modifying the first PPG signal and the second PPG signal. For example, the system 200 may use the temperature data to correct the wavelength disposition caused to the LED component by varying environmental and/or physiological temperature.

In some implementations, the system 200 may modify the first PPG signal and the second PPG signal based on pressure data (e.g., pressure measured at the measurement location). In some cases, one or more blood oxygen saturation metrics may be omitted (e.g., disregarded) based on unphysiological signal level changes that may cause blood oxygen saturation metrics to increase (e.g., spike). A change in blood oxygen saturation metrics may include a determined pattern. For example, the PPG signal associated with the red channel may include a first pattern when the blood oxygen saturation metrics decrease, and the PPG signal associated with the infrared channel may include a second pattern different than the first pattern when the blood oxygen saturation metrics decrease. The first pattern may include a decreasing slope, and the second pattern may include an increasing slope. The system 200 may determine that the first and second patterns may be absent from the respective PPG signals, and in such cases, the system 200 may omit the blood oxygen saturation metrics. In some cases, the blood oxygen saturation metrics may be compensated based on the rotation of a ring 104 relative to a user's finger. In such cases, a rotation detection method for determining a relative rotation of the ring 104 relative to a user's finger may be implemented. In such cases, the system 200 may receive multiple PPG signals and temperature signals (e.g., data), and may compensate (e.g., adjust, modify) determined blood oxygen saturation metrics based on a determined rotation of the ring 104.

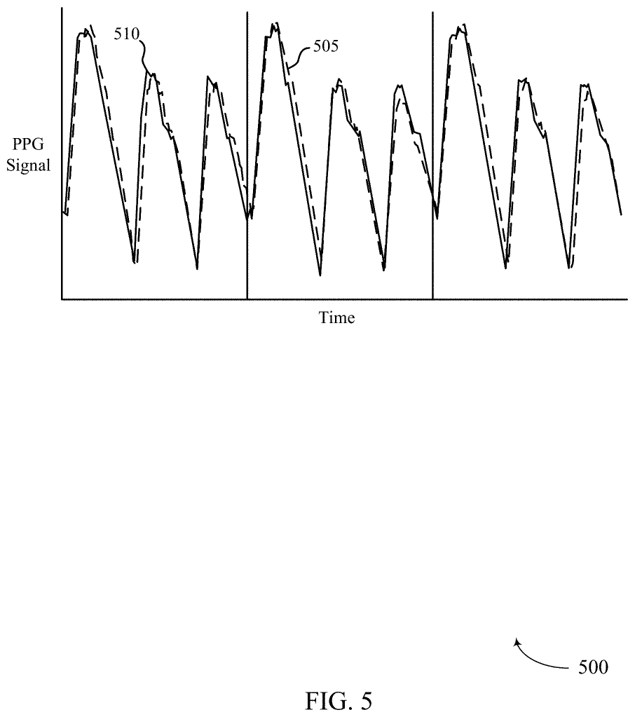

FIG. 5 illustrates an example of a timing diagram 500 that supports techniques for measuring blood oxygen levels in accordance with aspects of the present disclosure. The timing diagrams 500 may implement, or be implemented by, aspects of the system 100, system 200, wearable device diagram 300, or a combination thereof.

The timing diagram 500 may include a first PPG signal 505 and a second PPG signal 510. The first PPG signal 505 may be an example of a PPG signal from an infrared LED, and the second PPG signal 510 may be an example of a PPG signal from a red LED. In some cases, the red light associated with the red LED may be received at a PD faster than the PD receives an infrared light associated with the infrared LED. For example, in cases where a red LED 320-a and an infrared LED 320-b fire or activate simultaneously, the behavior of red and infrared light within a user's finger may be different due to physiological characteristics of the user's skin and other tissue, which may result in different arrival times of the red and infrared light at the respective PDs 325.

The timing diagram 500 may illustrate an example of peak matching where a peak point of the first PPG signal 505 may match (e.g., align with) a peak point of the second PPG signal 510. In such cases, the red LED and the infrared LED may be activated at a same time. The two PDs may receive the red light from the red LED at the same time and may receive the infrared light from the infrared LED at the same time. In some cases, a peak point of the first PPG signal 505 may be higher than a peak point of the second PPG signal 510 in which the shape of the first PPG signal 505 may vary from the shape of the second PPG signal 510. In some implementations, the system 200 may be configured to perform “peak matching” procedures in order to align the peaks of the respective PPG signals 505, 510 (e.g., align the peaks in the time domain) in order to produce more accurate and reliable blood oxygen measurements.

FIG. 6 illustrates an example of a GUI 600 that supports techniques for measuring blood oxygen levels in accordance with aspects of the present disclosure. The GUI 600 may implement, or be implemented by, aspects of the system 100, system 200, wearable device diagram 300, process flow 400, timing diagram 500 or any combination thereof. For example, the GUI 600 may be an example of a GUI 275 of a user device 106 (e.g., user device 106-a, 106-b, 106-c) corresponding to a user 102.

In some examples, the GUI 600 illustrates a series of application pages that may be displayed to a user 102 via the GUI 600 (e.g., GUI 275 illustrated in FIG. 2). In some implementations, the application page may display an indication of the one or more blood oxygen saturation metrics via alert 605. In such cases, the application page may include the alert 605 on the home page. In cases where a user's one or more blood oxygen saturation metrics may be determined, as described herein, the server 110 may transmit an alert 605 to the user 102, where the alert 605 is associated with the one or more blood oxygen saturation metrics for the user 102.

For example, the user 102 may receive an alert 605, which may indicate the one or more blood oxygen saturation metrics for the respective calendar day. The alerts 605 may be configurable/customizable, such that the user 102 may receive different alerts 605 based on the one or more blood oxygen saturation metrics.

In some cases, the user may take remedial action to address the one or more blood oxygen saturation metrics prior to the system 200 displaying the alert 605. In such cases, the system 200 may receive physiological data associated with the remedial action, and the system 200 may refrain from displaying the alert 605 (e.g., override the alert 605). In some examples, the system 200 may adjust the alert 605 based on receiving the physiological data associated with the remedial action.

As shown in FIG. 6, the application page may display information associated with determined blood oxygen saturation metrics via message 610. The user 102 may receive message 610, which may prompt the user 102 to verify or dismiss the message 610. In such cases, the application page may prompt the user 102 to confirm or dismiss the one or more blood oxygen saturation metrics. For example, the system 200 may receive, via the user device 106 and in response to determining the one or more blood oxygen saturation metrics, a confirmation of the one or more blood oxygen saturation metrics. Additionally, in some implementations, the application page may display one or more scores (e.g., Sleep Score, Readiness Score, Activity Score/activity goal progress) for the user 102 for the respective day.

The application pages may display a blood oxygen saturation metric card such as a “blood oxygen saturation metric confirmation card,” which indicates that the blood oxygen saturation metric has been recorded. Moreover, in some cases, the blood oxygen saturation metric may be used to update (e.g., modify) one or more scores associated with the user 102 (e.g., Sleep Score, Readiness Score). That is, data associated with the blood oxygen saturation metric may be used to update the scores for the user 102 for the following calendar day. In some cases, the Readiness Score may be updated based on the blood oxygen saturation metric.

In some cases, the messages 610 displayed to the user 102 via the GUI 600 of the user device 106 may indicate how the blood oxygen saturation metric affected the overall scores (e.g., overall Readiness Score) and/or the individual contributing factors. For example, a message may indicate “It looks like your body is under strain right now, but if you're feeling okay, doing a breathing exercise or meditation can help improve your blood oxygen saturation metric.” In cases where the blood oxygen saturation metric was not optimal, the messages 610 may provide suggestions for the user in order to improve their general health. The GUI 600 may indicate one or more parameters of the blood oxygen saturation metric, including a temperature, heart rate, HRV, and the like.

In some cases, the user 102 may log symptoms via input 615. For example, the system 200 may receive user input (e.g., tags) to log symptoms associated with the blood oxygen saturation metric. In some examples, the system 200 may receive supplemental data such as alcohol intake, stress, anxiety, wake-ups in the middle of the night, and the like. The system 200 may recommend tags to the user 102 based on user history and the blood oxygen saturation metric.

The GUI 600 may also include messages 610 that include insights, recommendations, and the like associated with the blood oxygen saturation metric. The server 110 of system 200 may cause the GUI 600 of the user device 106 to display a message 610 associated with the blood oxygen saturation metric. The user device 106 may display recommendations and/or information associated with the blood oxygen saturation metric via message 610.

As noted previously herein, a determined blood oxygen saturation metric may be beneficial to a user's overall health by providing metrics to the user that may enable the user to understand how behavior changes (e.g., improvements in sleep, exercise, diet, and mood) may help improve the user's blood oxygen saturation metric. In some implementations, the user device 106 and/or servers 110 may generate alerts 605 associated with the blood oxygen saturation metric that may be displayed to the user via the GUI 600. In some cases, the alert 605 may display a recommendation of how the user may adjust their lifestyle to improve the blood oxygen saturation metric. In some examples, the system 200 may recommend a guided mediation or breathing exercise for the user 102 after determining the blood oxygen saturation metric.

In some implementations, the system 200 may provide additional insight regarding the user's blood oxygen saturation metric. For example, the application pages may indicate one or more physiological parameters (e.g., contributing factors) that contributed to the user's blood oxygen saturation metric. In other words, the system 200 may be configured to provide some information or other insights regarding the blood oxygen saturation metric. Personalized insights may indicate aspects of collected physiological data (e.g., contributing factors within the physiological data) that were used to generate the blood oxygen saturation metric.

In some implementations, the system 200 may be configured to receive user inputs regarding the blood oxygen saturation metric in order to train classifiers (e.g., supervised learning for a machine learning classifier) and improve blood oxygen saturation metric determination techniques.

In some cases, the system 200 may be configured to detect apnea based on the determined blood oxygen saturation metrics and display the apnea prediction to the user via GUI 600. In some examples, the system 200 may display breathing insights, overnight variations associated with the blood oxygen saturation metric, and blood oxygen saturation metric trends, via message 610. For example, the system 200 may display a timing diagram that may include values of the blood oxygen saturation metric overnight. In such cases, the system 200 (e.g., a wearable ring device) may determine the overnight variation based on the values of the blood oxygen saturation metric and display this variation to the user 102 via GUI 600 (e.g., based on the overnight variation exceeding a threshold value). In some examples, the system 200 may determine a variability associated with the blood oxygen saturation metric and display the variability to the user 102 via message 610 or alert 605. For example, the alert 605 may include a sleep disturbance alert.

In some aspects, the system 200 may be configured to generate or calculate a “risk score” associated with one or more medical conditions based on the determined blood oxygen saturation metrics. For example, in some cases, the system 200 may calculate a sleep apnea risk metric associated with the user based on the blood oxygen saturation metrics. In this example, the sleep apnea risk metric may be associated with a relative probability that the user has experienced symptoms of sleep apnea, or will experience symptoms of sleep apnea in the future. In some cases, the system 200 may use one or more machine learning models (e.g., machine learning classifiers, Random Forest algorithms, neural networks, etc.) to calculate sleep apnea risk metrics. For example, the system 200 may input the blood oxygen saturation metrics and additional physiological data (e.g., respiration rate, HRV, etc.) into a machine learning model, where the machine learning model is configured to calculate a sleep apnea risk metric (and/or additional risk metrics for other medical conditions) based on the inputted data.

In some cases, sleep apnea risk metrics may be displayed to the user, such as via a GUI of the user device. Additionally, or alternatively, the system 200 may generate messages or alerts based on the sleep apnea risk metrics. For example, if the sleep apnea risk metric is above a threshold (suggesting that the user has likely experienced sleep apnea), the system 200 may cause the user device to display a message suggesting that the user talk to their doctor about sleep apnea. Other messages/alerts that may be generated based on sleep apnea risk metrics may include, but are not limited to, links to reading materials and other content associated with sleep apnea, alerts to the user's physician or other administrator, and the like.

In some examples, a wearable ring device of the system 200 may determine the overnight variation based on the values of the blood oxygen saturation metric and may refrain from displaying the variation to the user 102 via the GUI 600 based on the overnight variation falling below the threshold value. That is, the wearable ring device may determine that the overnight variation is less than the threshold value and may transmit, to a user device of the system 200, an indication that the variation is less than the threshold (e.g., trend_stable). That is, the wearable ring device may refrain from transmitting, to the user device, an indication of the one or more blood oxygen saturation metrics based on transmitting the indication that the overnight variation is less than the threshold. In other words, the wearable ring device may refrain from transmitting blood oxygen saturation metrics to the user device in cases where the user's blood oxygen saturation has not changed substantially. Such techniques may reduce power consumption at the wearable ring device and the user device, and therefore improve battery performance. In such cases, the wearable ring device may transmit the indication that the variation is less than the threshold periodically (e.g., every 1 minute or so) during a time interval that the variation is less than the threshold.

Conversely, as described previously, the wearable ring device may transmit, to the user device, an indication of the one or more blood oxygen saturation metrics based on the overnight variation exceeding the threshold. In other words, the wearable device may transmit blood oxygen saturation metrics to the user device in cases where the user's blood oxygen levels have changed substantially (e.g., more than a threshold amount). In such cases, the wearable ring device may transmit the indication of the one or more blood oxygen saturation metrics periodically (e.g., at a predefined frequency) during a time interval that the overnight variation exceeds the threshold. Transmitting the indication of the one or more blood oxygen saturation based on the overnight variation exceeding the threshold may result in increased data savings (e.g., 60-90%), increased battery savings (e.g., 0.5-1 day per cycle), and reduced data sync durations (e.g., morning sync is 2× faster).

Though described in the context of an overnight variation, this is not to be regarded as a limitation of the present disclosure. In this regard, the system 200 (e.g., the wearable ring device) may determine a variation over any time interval, including but not limited to an overnight time interval, a daytime time interval, and the like, with respect to the techniques described herein.

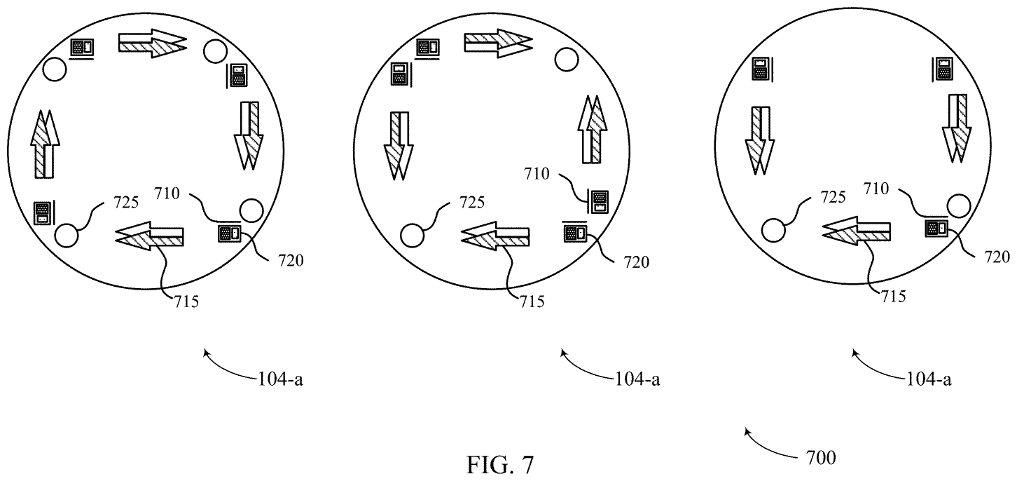

FIG. 7 illustrates an example of a wearable device diagram 700 that supports techniques for measuring blood oxygen levels in accordance with aspects of the present disclosure. The wearable device diagram 700 may implement, or be implemented by, aspects of the system 100, system 200, wearable device diagram 300, process flow 400, GUI 600, or a combination thereof. For example, wearable device diagram 700 may illustrate examples of wearable devices 104-a, 104-b, and 104-c as described with reference to FIGS. 1-6. Although the wearable devices may be described as a wearable ring device in FIG. 7, aspects and components of the wearable devices illustrated in FIG. 7 may be implemented in any type of wearable device (e.g., a watch, a bracelet, a necklace, and the like).

The wearable device diagram 700 may include a wearable device 104-a including one or more light guides 710, primary light direction 715, one or more LEDs 720, and one or more photodiodes 725. The LED 720 may emit light that is transmitted in the primary light direction 715 to the photodiode 725. The light guide 710 may be used to direct the light in the primary light direction 715. The primary light direction 715 may be from the LED 720, through the tissue of the user, and to the photodiode 725. In some cases, an optimal quantity of LED 720 and photodiode 725 pairs may be three pairs, four pairs, or five pairs.

In some cases, the LEDs 720 may be an example of a laser. The laser may be an example of a photonic crystal surface emitting laser diode (PCSEL) or a vertical cavity surface emitting laser (VCSEL). In some cases, the LED 720 may be an example of an edge emitting LED. By using a laser rather than the LED 720, the system 200 may eliminate the use of the light guide 710. The laser diode may provide a stream of light with increased concentration, accuracy, and precision.

The light source (e.g., LED 720) may use light with a wavelength of 740 to 760 nm. In such cases, the wavelength of the light may be outside of the visible light spectrum such that the wearable device 104-a may emit fewer visual disturbances caused by tissue scattering and light leakage. By using the laser as a light source, the wavelength distribution of the wearable device 104-a may be tighter and adjusted to the 760 nm peak value. In some cases, using a laser diode may prevent light leakage to the visual wavelength spectrum. The wavelength of 740 to 760 nm may provide a similar penetration to the skin of the user as compared to the wavelength of infrared light, thereby enabling improved blood oxygen saturation measurements.

In some aspects, a wearable device 104 may include different quantities and/or arrangements of light sources and photodiodes/photodetectors. For example, as shown in the wearable device 104-b illustrated in FIG. 7, the wearable device 104-b may include four LEDs 720 and two photodiodes 725. Similarly, as shown in the wearable device 104-c, the wearable device 104-c may include three LEDs 720 and two photodiodes 725. In this regard, the wearable device 104-b may include two LEDs 720 for each photodiode 725 (2:1 ratio) and the wearable device 104-c may include three LEDs 720 for every two photodiodes 725 (3:2 ratio), as compared to the wearable device 104-a, which includes one LED 720 for each photodiode 725 (1:1 ratio). In some implementations, as shown in the wearable device 104-b, multiple LEDs 720 may be positioned adjacent to one another, where light guides 710 are disposed between the LEDs 720 in order to direct the light from the LEDs 720 along the primary light directions 715 of the respective LEDs 720.

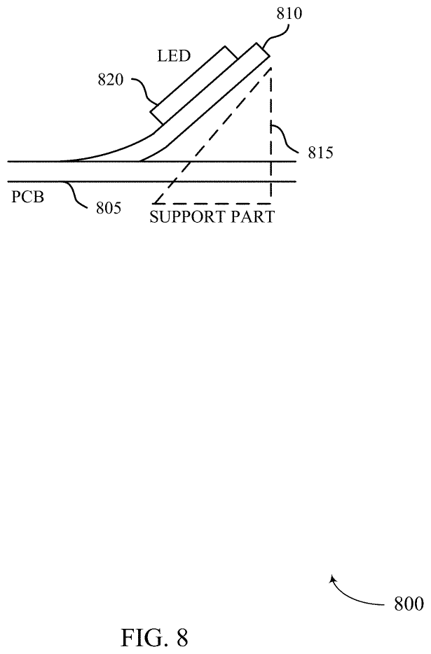

FIG. 8 illustrates an example of a wearable device diagram 800 that supports techniques for measuring blood oxygen levels in accordance with aspects of the present disclosure. The wearable device diagram 800 may implement, or be implemented by, aspects of the system 100, system 200, wearable device diagram 300, process flow 400, GUI 600, wearable device diagram 700, or a combination thereof. For example, wearable device diagram 800 may illustrate examples of wearable devices 104 as described with reference to FIGS. 1-7. Although the wearable devices may be described as a ring in FIG. 8, aspects and components of the wearable devices illustrated in FIG. 8 may be implemented in any type of wearable device (e.g., a watch, a bracelet, a necklace, and the like).

The wearable device diagram 800 may include a PCB 805, a support 810, an angle 815, and an LED 820. The LED 820 may be mounted (e.g., attached) to the support 810. The support 810 may be mounted to the PCB 805. The support 810 may include the light source (e.g., LED 820) to provide an optimal position for the light primary direction. For example, the support 810 may be positioned at an angle 815 relative to an axis of the PCB 805. In such cases, the LED 820 attached to the support 810 may emit light such that the light may not be transmitted directly through the tissue (e.g., finger). For example, the LED 820 may be angled towards the PDs. The blood oxygen measurement may be improved based on angling the support 810 (e.g., and the corresponding LED 820) towards the PD at the angle 815.

The PCB 805 may be formed and bent to allow optimal light direction to the photodiode through the tissue of the user. The optimal light direction may increase a signal-to-noise ratio and decrease motion influences on the blood oxygen measurement. For example, the first set of PPG sensors (e.g., LED 820) may be angled towards a first set of one or more PDs. The second set of PPG sensors (e.g., LED 820) may be angled towards a second set of one or more PDs.

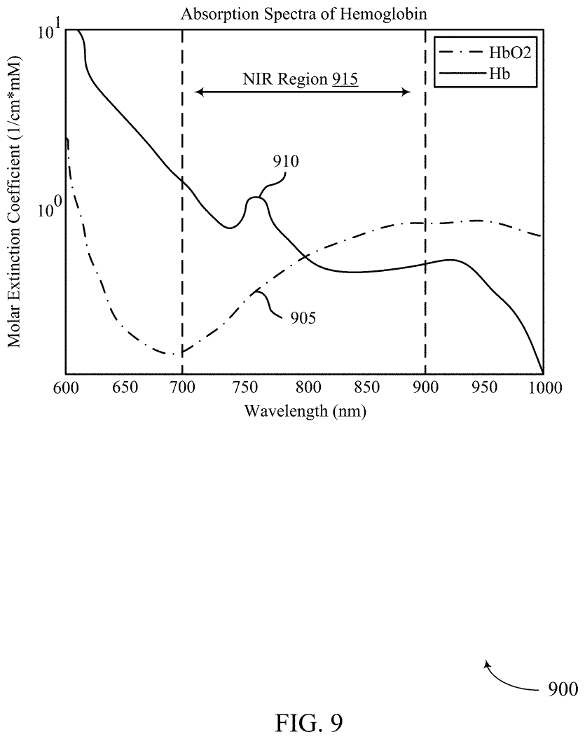

FIG. 9 illustrates an example of a frequency diagram 900 that supports techniques for measuring blood oxygen levels in accordance with aspects of the present disclosure. The frequency diagram 900 may implement, or be implemented by, aspects of the system 100, system 200, wearable device diagram 300, process flow 400, GUI 600, wearable device diagram 700, wearable device diagram 800, or a combination thereof.

The frequency diagram 900 may be an example of an absorption spectra of hemoglobin. For example, the frequency diagram 900 may include an oxyhemoglobin curve 905 and a hemoglobin curve 910. The near infrared (NIR) region 915 may extend from a wavelength of 700 nm to 900 nm. The hemoglobin curve 910 may experience a peak at a wavelength of approximately 760 nm. In such cases, the peak of the hemoglobin curve 910 may be experienced between a wavelength of 740 nm and 770 nm. The system 200 may receive PPG signals with accurate amplitude relation when comparing the photodiode responses with wavelengths of 740 to 770 nm and 940 nm wavelength LEDs or lasers. By using a 760 nm light source, rather than a red light source, light penetration depth in the skin of the user may be more uniform between the respective PPG channels. In some cases, a 940 nm light source may produce similar effects to the 760 nm light source such that the penetration depths may be closer to each other.

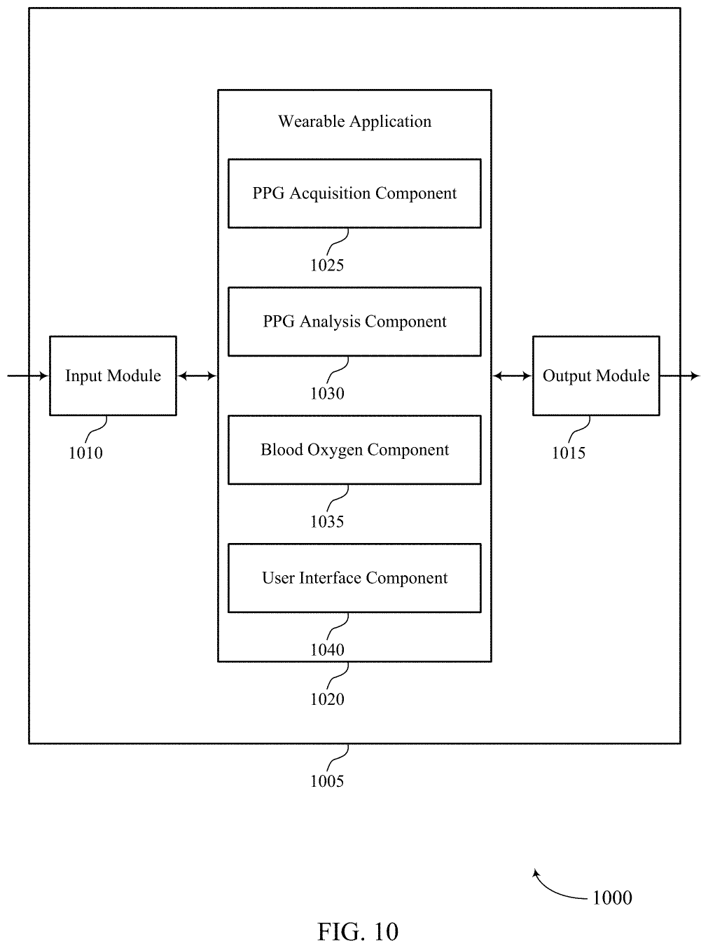

FIG. 10 shows a block diagram 1000 of a device 1005 that supports techniques for measuring blood oxygen levels in accordance with aspects of the present disclosure. The device 1005 may include an input module 1010, an output module 1015, and a wearable application 1020. The device 1005 may also include a processor. Each of these components may be in communication with one another (e.g., via one or more buses).

The input module 1010 may provide a means for receiving information such as packets, user data, control information, or any combination thereof associated with various information channels (e.g., control channels, data channels, information channels related to illness detection techniques). Information may be passed on to other components of the device 1005. The input module 1010 may utilize a single antenna or a set of multiple antennas.

The output module 1015 may provide a means for transmitting signals generated by other components of the device 1005. For example, the output module 1015 may transmit information such as packets, user data, control information, or any combination thereof associated with various information channels (e.g., control channels, data channels, information channels related to illness detection techniques). In some examples, the output module 1015 may be co-located with the input module 1010 in a transceiver module. The output module 1015 may utilize a single antenna or a set of multiple antennas.

For example, the wearable application 1020 may include a PPG acquisition component 1025, a PPG analysis component 1030, a blood oxygen component 1035, a user interface component 1040, or any combination thereof. In some examples, the wearable application 1020, or various components thereof, may be configured to perform various operations (e.g., receiving, monitoring, transmitting) using or otherwise in cooperation with the input module 1010, the output module 1015, or both. For example, the wearable application 1020 may receive information from the input module 1010, send information to the output module 1015, or be integrated in combination with the input module 1010, the output module 1015, or both to receive information, transmit information, or perform various other operations as described herein.

The PPG acquisition component 1025 may be configured as or otherwise support a means for receiving, via a wearable device, a first PPG signal for a user acquired during a time interval using a first set of PPG sensors. The PPG acquisition component 1025 may be configured as or otherwise support a means for receiving, via the wearable device, a second PPG signal for the user acquired during the time interval using a second set of PPG sensors that are different from the first set of PPG sensors. The PPG analysis component 1030 may be configured as or otherwise support a means for comparing the first PPG signal and the second PPG signal. The blood oxygen component 1035 may be configured as or otherwise support a means for determining one or more blood oxygen saturation metrics for the user during the time interval based at least in part on the comparison of the first PPG signal and the second PPG signal. The user interface component 1040 may be configured as or otherwise support a means for causing a GUI to display an indication of the one or more blood oxygen saturation metrics.

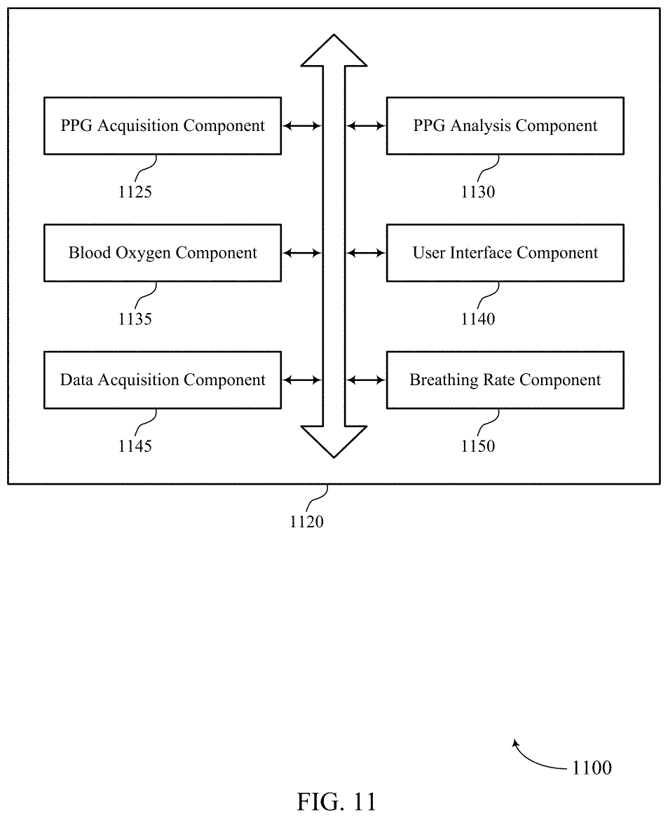

FIG. 11 shows a block diagram 1100 of a wearable application 1120 that supports techniques for measuring blood oxygen levels in accordance with aspects of the present disclosure. The wearable application 1120 may be an example of aspects of a wearable application or a wearable application 1020, or both, as described herein. The wearable application 1120, or various components thereof, may be an example of means for performing various aspects of techniques for measuring blood oxygen levels as described herein. For example, the wearable application 1120 may include a PPG acquisition component 1125, a PPG analysis component 1130, a blood oxygen component 1135, a user interface component 1140, a data acquisition component 1145, a breathing rate component 1150, or any combination thereof. Each of these components may communicate, directly or indirectly, with one another (e.g., via one or more buses).

The PPG acquisition component 1125 may be configured as or otherwise support a means for receiving, via a wearable device, a first PPG signal for a user acquired during a time interval using a first set of PPG sensors. In some examples, the PPG acquisition component 1125 may be configured as or otherwise support a means for receiving, via the wearable device, a second PPG signal for the user acquired during the time interval using a second set of PPG sensors that are different from the first set of PPG sensors. The PPG analysis component 1130 may be configured as or otherwise support a means for comparing the first PPG signal and the second PPG signal. The blood oxygen component 1135 may be configured as or otherwise support a means for determining one or more blood oxygen saturation metrics for the user during the time interval based at least in part on the comparison of the first PPG signal and the second PPG signal. The user interface component 1140 may be configured as or otherwise support a means for causing a GUI to display an indication of the one or more blood oxygen saturation metrics.

In some examples, to support receiving the first PPG signal and the second PPG signal, the PPG acquisition component 1125 may be configured as or otherwise support a means for receiving a first PPG measurement utilizing a first channel between a first subset of the first set of PPG sensors. In some examples, to support receiving the first PPG signal and the second PPG signal, the PPG acquisition component 1125 may be configured as or otherwise support a means for receiving a second PPG measurement utilizing a second channel between a second subset of the first set of PPG sensors, wherein the first PPG signal is based at least in part on the first PPG measurement and the second PPG measurement. In some examples, to support receiving the first PPG signal and the second PPG signal, the PPG acquisition component 1125 may be configured as or otherwise support a means for receiving a third PPG measurement utilizing a third channel between a first subset of the second set of PPG sensors. In some examples, to support receiving the first PPG signal and the second PPG signal, the PPG acquisition component 1125 may be configured as or otherwise support a means for receiving a fourth PPG measurement utilizing a fourth channel between a second subset of the second set of PPG sensors, wherein the second PPG signal is based at least in part on the third PPG measurement and the fourth PPG measurement.

In some examples, the first set of PPG sensors comprises a first light source, a first photodetector, and a second photodetector. In some examples, the first channel comprises a channel between the first light source and the first photodetector. In some examples, the second channel comprises a channel between the first light source and the second photodetector. In some examples, the second set of PPG sensors comprises a second light source, the first photodetector, and the second photodetector. In some examples, the third channel comprises a channel between the second light source and the first photodetector. In some examples, the fourth channel comprises a channel between the second light source and the second photodetector. In some examples, the second light source is configured to generate light with a different wavelength as compared to light generated via the first light source.

In some examples, to support receiving the first PPG signal and the second PPG signal, the PPG acquisition component 1125 may be configured as or otherwise support a means for selectively controlling an activation state of the first light source and the second light source such that the first light source and the second light source are simultaneously in an active activation state.

In some examples, to support receiving the first PPG signal and the second PPG signal, the PPG acquisition component 1125 may be configured as or otherwise support a means for sequentially controlling an activation state of the first light source and the second light source such that the first light source is in an active activation state when the second light source is in an inactive activation state, and vice versa.

In some examples, the PPG analysis component 1130 may be configured as or otherwise support a means for combining the first PPG measurement and the second PPG measurement to generate the first PPG signal. In some examples, the PPG analysis component 1130 may be configured as or otherwise support a means for combining the third PPG measurement and the fourth PPG measurement to generate the second PPG signal.

In some examples, the first PPG measurement and the second PPG measurement and the third PPG measurement and the fourth PPG measurement, respectively, are combined using one or more mathematical operations, the one or more mathematical operations comprising an averaging operation, a weighted averaging operation, or both.

In some examples, the PPG acquisition component 1125 may be configured as or otherwise support a means for receiving a third PPG signal for the user acquired during the time interval using a third set of PPG sensors that are different from the first set of PPG sensors and the second set of PPG sensors.

In some examples, to support receiving the third PPG signal, the PPG acquisition component 1125 may be configured as or otherwise support a means for receiving a fifth PPG measurement utilizing a fifth channel between a first subset of the third set of PPG sensors. In some examples, to support receiving the third PPG signal, the PPG acquisition component 1125 may be configured as or otherwise support a means for receiving a sixth PPG measurement utilizing a sixth channel between a second subset of the third set of PPG sensors, wherein the third PPG signal is based at least in part on the fifth PPG measurement and the sixth PPG measurement.

In some examples, the first set of PPG sensors, the second set of PPG sensors, and the third set of PPG sensors use three different wavelengths.

In some examples, the PPG analysis component 1130 may be configured as or otherwise support a means for determining a ratio between the first PPG signal and the second PPG signal, wherein determining the one or more blood oxygen saturation metrics is based at least in part on the ratio of the first PPG signal and the second PPG signal.

In some examples, the PPG analysis component 1130 may be configured as or otherwise support a means for determining a first perfusion index between a first amplitude and a first baseline amplitude level of the first PPG signal. In some examples, the PPG analysis component 1130 may be configured as or otherwise support a means for determining a second perfusion index between a second amplitude and a second baseline amplitude level of the second PPG signal, wherein determining the ratio of the first PPG signal and the second PPG signal comprises determining a ratio between the first perfusion index and the second perfusion index.

In some examples, the PPG analysis component 1130 may be configured as or otherwise support a means for generating a blood oxygen saturation signal based at least in part on the comparison of the first PPG signal and the second PPG signal. In some examples, the PPG analysis component 1130 may be configured as or otherwise support a means for identifying one or more calibration coefficients for blood oxygen saturation calculations, wherein the one or more blood oxygen saturation metrics are determined based at least in part on the blood oxygen saturation signal and the one or more calibration coefficients.

In some examples, the data acquisition component 1145 may be configured as or otherwise support a means for acquiring temperature data during the time interval. In some examples, the PPG analysis component 1130 may be configured as or otherwise support a means for modifying the first PPG signal and the second PPG signal based at least in part on the temperature data, wherein determining the one or more blood oxygen saturation metrics is based at least in part on modifying the first PPG signal and the second PPG signal.

In some examples, the first set of PPG sensors comprises a first light source and a first set of one or more photodetectors. In some examples, the second set of PPG sensors comprises a second light source and a second set of one or more photodetectors. In some examples, each of the first light source, the second light source, the first set of one or more photodetectors, and the second set of one or more photodetectors are positioned at different radial positions relative to an axis of the wearable device and along an inner circumference of the wearable device.

In some examples, the data acquisition component 1145 may be configured as or otherwise support a means for receiving physiological data associated with the user via the wearable device, the physiological data comprising at least motion data or temperature data, or both, wherein receiving the first PPG signal, the second PPG signal, or both, is based at least in part on the motion data satisfying a threshold motion metric and the temperature data satisfying a threshold temperature metric.

In some examples, the breathing rate component 1150 may be configured as or otherwise support a means for determining a breathing rate for the user based at least in part on the motion data, the first PPG signal, the second PPG signal, or a combination thereof. In some examples, the blood oxygen component 1135 may be configured as or otherwise support a means for updating the one or more blood oxygen saturation metrics for the user based at least in part on determining the breathing rate for the user.

In some examples, the motion data satisfies the threshold motion metric if the motion data is less than or equal to the threshold motion metric. In some examples, the temperature data satisfies the threshold temperature metric if the temperature is greater than or equal to the threshold temperature metric.

In some examples, the data acquisition component 1145 may be configured as or otherwise support a means for receiving physiological data associated with the user via the wearable device, the physiological data comprising at least heart rate data or PPG signal feature data, or both, wherein receiving the first PPG signal, the second PPG signal, or both, is based at least in part on the heart rate satisfying a threshold heart rate metric and the PPG signal feature data satisfying a threshold metric.

In some examples, the blood oxygen component 1135 may be configured as or otherwise support a means for determining a variation of the one or more blood oxygen saturation metrics for the user during the time interval. In some examples, the blood oxygen component 1135 may be configured as or otherwise support a means for transmitting, to a user device, an indication of the one or more blood oxygen saturation metrics based at least in part on the variation exceeding a threshold, wherein displaying an indication of the one or more blood oxygen saturation metrics is based at least in part on the transmitting

In some examples, the user interface component 1140 may be configured as or otherwise support a means for causing the graphical user interface to display a sleep disturbance alert based at least in part on the variation of the one or more blood oxygen saturation metrics exceeding the threshold.

In some examples, at least one photodetector is included within both the first set of PPG sensors and the second set of PPG sensors.

In some examples, the first PPG signal, the second PPG signal, or both, are based at least in part on light that is transmitted through tissue of the user and light that is reflected by the tissue of the user.

In some examples, the first set of PPG sensors are configured to acquire the first PPG signal using light of a first wavelength. In some examples, the second set of PPG sensors are configured to acquire the second PPG signal using light of a second wavelength that is different from the first wavelength.

In some examples, the first set of PPG sensors comprise at least one red light-emitting diode. In some examples, the second set of PPG sensors comprise at least one infrared light-emitting diode.

In some examples, the at least one red light-emitting diode emits light of 740-760 nm wavelength.

In some examples, the first set of PPG sensors comprise at least one laser diode. In some examples, the second set of PPG sensors comprise at least one laser diode.

In some examples, the first set of PPG sensors are angled towards a first set of one or more photodetectors. In some examples, the second set of PPG sensors are angled towards a second set of one or more photodetectors.

In some examples, the first PPG signal, the second PPG signal, or both, are acquired from the user based on arterial blood flow.

In some examples, the wearable device comprises a wearable ring device.

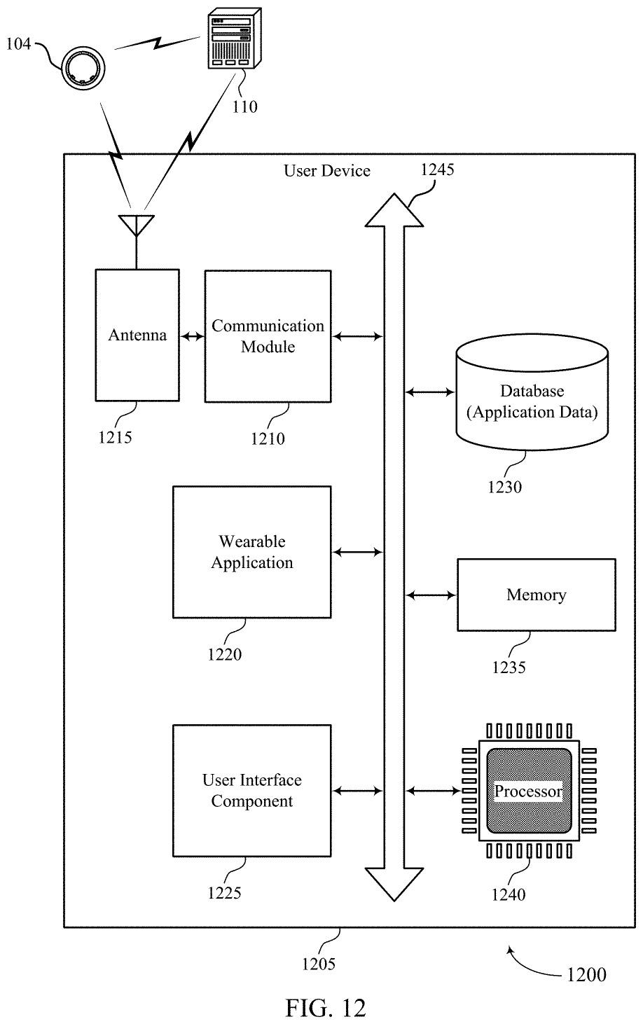

FIG. 12 shows a diagram of a system 1200 including a device 1205 that supports techniques for measuring blood oxygen levels in accordance with aspects of the present disclosure. The device 1205 may be an example of or may include the components of a device 1005 as described herein. The device 1205 may include an example of a user device 106, as described previously herein. The device 1205 may include components for bi-directional communications including components for transmitting and receiving communications with a wearable device 104 and a server 110, such as a wearable application 1220, a communication module 1210, an antenna 1215, a user interface component 1225, a database (application data) 1230, a memory 1235, and a processor 1240. These components may be in electronic communication or otherwise coupled (e.g., operatively, communicatively, functionally, electronically, electrically) via one or more buses (e.g., a bus 1245).

The communication module 1210 may manage input and output signals for the device 1205 via the antenna 1215. The communication module 1210 may include an example of the communication module 220-b of the user device 106 shown and described in FIG. 2. In this regard, the communication module 1210 may manage communications with the ring 104 and the server 110, as illustrated in FIG. 2. The communication module 1210 may also manage peripherals not integrated into the device 1205. In some cases, the communication module 1210 may represent a physical connection or port to an external peripheral. In some cases, the communication module 1210 may utilize an operating system such as iOS®, ANDROID®, MS-DOS®, MS-WINDOWS®, OS/2®, UNIX®, LINUX®, or another known operating system. In other cases, the communication module 1210 may represent or interact with a wearable device (e.g., ring 104), modem, a keyboard, a mouse, a touchscreen, or a similar device. In some cases, the communication module 1210 may be implemented as part of the processor 1240. In some examples, a user may interact with the device 1205 via the communication module 1210, user interface component 1225, or via hardware components controlled by the communication module 1210.

In some cases, the device 1205 may include a single antenna 1215. However, in some other cases, the device 1205 may have more than one antenna 1215, which may be capable of concurrently transmitting or receiving multiple wireless transmissions. The communication module 1210 may communicate bi-directionally, via the one or more antennas 1215, wired, or wireless links as described herein. For example, the communication module 1210 may represent a wireless transceiver and may communicate bi-directionally with another wireless transceiver. The communication module 1210 may also include a modem to modulate the packets, to provide the modulated packets to one or more antennas 1215 for transmission, and to demodulate packets received from the one or more antennas 1215.

The user interface component 1225 may manage data storage and processing in a database 1230. In some cases, a user may interact with the user interface component 1225. In other cases, the user interface component 1225 may operate automatically without user interaction. The database 1230 may be an example of a single database, a distributed database, multiple distributed databases, a data store, a data lake, or an emergency backup database.

The memory 1235 may include RAM and ROM. The memory 1235 may store computer-readable, computer-executable software including instructions that, when executed, cause the processor 1240 to perform various functions described herein. In some cases, the memory 1235 may contain, among other things, a BIOS which may control basic hardware or software operation such as the interaction with peripheral components or devices.

The processor 1240 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 1240 may be configured to operate a memory array using a memory controller. In other cases, a memory controller may be integrated into the processor 1240. The processor 1240 may be configured to execute computer-readable instructions stored in a memory 1235 to perform various functions (e.g., functions or tasks supporting a method and system for sleep staging algorithms).

For example, the wearable application 1220 may be configured as or otherwise support a means for receiving, via a wearable device, a first PPG signal for a user acquired during a time interval using a first set of PPG sensors. The wearable application 1220 may be configured as or otherwise support a means for receiving, via the wearable device, a second PPG signal for the user acquired during the time interval using a second set of PPG sensors that are different from the first set of PPG sensors. The wearable application 1220 may be configured as or otherwise support a means for comparing the first PPG signal and the second PPG signal. The wearable application 1220 may be configured as or otherwise support a means for determining one or more blood oxygen saturation metrics for the user during the time interval based at least in part on the comparison of the first PPG signal and the second PPG signal. The wearable application 1220 may be configured as or otherwise support a means for causing a GUI to display an indication of the one or more blood oxygen saturation metrics.

By including or configuring the wearable application 1220 in accordance with examples as described herein, the device 1205 may support techniques for improved blood oxygen measurements.

The wearable application 1220 may include an application (e.g., “app”), program, software, or other component that is configured to facilitate communications with a ring 104, server 110, other user devices 106, and the like. For example, the wearable application 1220 may include an application executable on a user device 106 that is configured to receive data (e.g., physiological data) from a ring 104, perform processing operations on the received data, transmit and receive data with the servers 110, and cause presentation of data to a user 102.

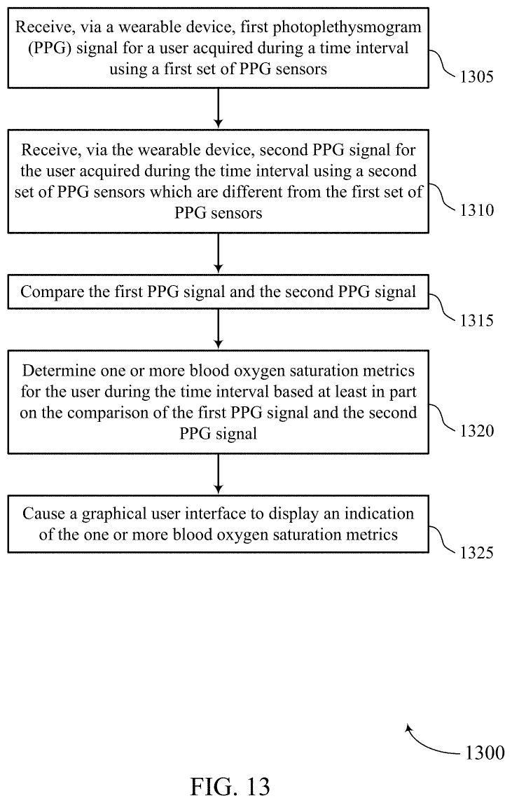

FIG. 13 shows a flowchart illustrating a method 1300 that supports techniques for measuring blood oxygen levels in accordance with aspects of the present disclosure. The operations of the method 1300 may be implemented by a user device or its components as described herein. For example, the operations of the method 1300 may be performed by a user device as described with reference to FIGS. 1 through 12. In some examples, a user device may execute a set of instructions to control the functional elements of the user device to perform the described functions. Additionally or alternatively, the user device may perform aspects of the described functions using special-purpose hardware.

At 1305, the method may include receiving, via a wearable device, a first PPG signal for a user acquired during a time interval using a first set of PPG sensors. The operations of 1305 may be performed in accordance with examples as disclosed herein. In some examples, aspects of the operations of 1305 may be performed by a PPG acquisition component 1125 as described with reference to FIG. 11.

At 1310, the method may include receiving, via the wearable device, a second PPG signal for the user acquired during the time interval using a second set of PPG sensors that are different from the first set of PPG sensors. The operations of 1310 may be performed in accordance with examples as disclosed herein. In some examples, aspects of the operations of 1310 may be performed by a PPG acquisition component 1125 as described with reference to FIG. 11.

At 1315, the method may include comparing the first PPG signal and the second PPG signal. The operations of 1315 may be performed in accordance with examples as disclosed herein. In some examples, aspects of the operations of 1315 may be performed by a PPG analysis component 1130 as described with reference to FIG. 11.

At 1320, the method may include determining one or more blood oxygen saturation metrics for the user during the time interval based at least in part on the comparison of the first PPG signal and the second PPG signal. The operations of 1320 may be performed in accordance with examples as disclosed herein. In some examples, aspects of the operations of 1320 may be performed by a blood oxygen component 1135 as described with reference to FIG. 11.

At 1325, the method may include causing a GUI to display an indication of the one or more blood oxygen saturation metrics. The operations of 1325 may be performed in accordance with examples as disclosed herein. In some examples, aspects of the operations of 1325 may be performed by a user interface component 1140 as described with reference to FIG. 11.

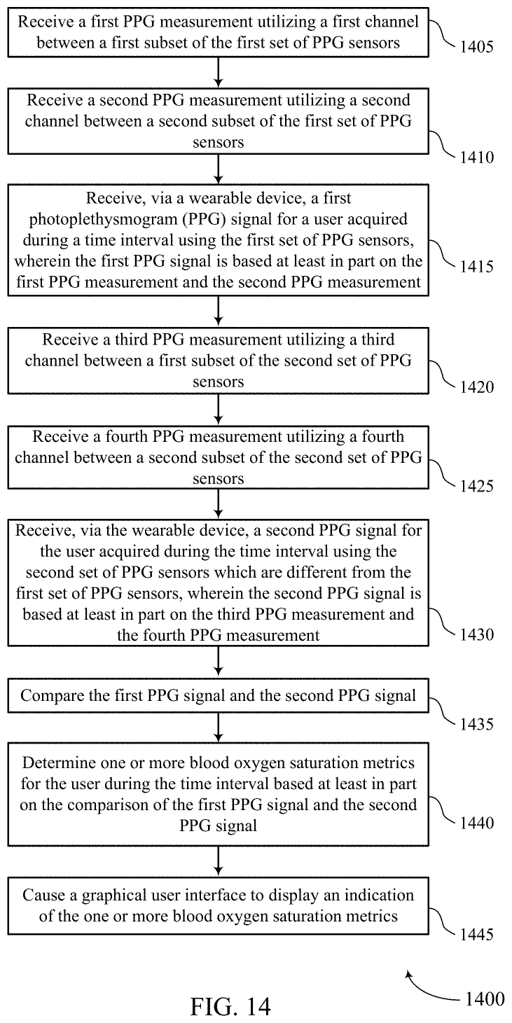

FIG. 14 shows a flowchart illustrating a method 1400 that supports techniques for measuring blood oxygen levels in accordance with aspects of the present disclosure. The operations of the method 1400 may be implemented by a user device or its components as described herein. For example, the operations of the method 1400 may be performed by a user device as described with reference to FIGS. 1 through 12. In some examples, a user device may execute a set of instructions to control the functional elements of the user device to perform the described functions. Additionally or alternatively, the user device may perform aspects of the described functions using special-purpose hardware.

At 1405, the method may include receiving a first PPG measurement utilizing a first channel between a first subset of the first set of PPG sensors. The operations of 1405 may be performed in accordance with examples as disclosed herein. In some examples, aspects of the operations of 1405 may be performed by a PPG acquisition component 1125 as described with reference to FIG. 11.

At 1410, the method may include receiving a second PPG measurement utilizing a second channel between a second subset of the first set of PPG sensors. The operations of 1410 may be performed in accordance with examples as disclosed herein. In some examples, aspects of the operations of 1410 may be performed by a PPG acquisition component 1125 as described with reference to FIG. 11.

At 1415, the method may include receiving, via a wearable device, a first PPG signal for a user acquired during a time interval using the first set of PPG sensors, wherein the first PPG signal is based at least in part on the first PPG measurement and the second PPG measurement. The operations of 1415 may be performed in accordance with examples as disclosed herein. In some examples, aspects of the operations of 1415 may be performed by a PPG acquisition component 1125 as described with reference to FIG. 11.

At 1420, the method may include receiving a third PPG measurement utilizing a third channel between a first subset of the second set of PPG sensors. The operations of 1420 may be performed in accordance with examples as disclosed herein. In some examples, aspects of the operations of 1420 may be performed by a PPG acquisition component 1125 as described with reference to FIG. 11.

At 1425, the method may include receiving a fourth PPG measurement utilizing a fourth channel between a second subset of the second set of PPG sensors. The operations of 1425 may be performed in accordance with examples as disclosed herein. In some examples, aspects of the operations of 1425 may be performed by a PPG acquisition component 1125 as described with reference to FIG. 11.

At 1430, the method may include receiving, via the wearable device, a second PPG signal for the user acquired during the time interval using the second set of PPG sensors that are different from the first set of PPG sensors, wherein the second PPG signal is based at least in part on the third PPG measurement and the fourth PPG measurement. The operations of 1430 may be performed in accordance with examples as disclosed herein. In some examples, aspects of the operations of 1430 may be performed by a PPG acquisition component 1125 as described with reference to FIG. 11.

At 1435, the method may include comparing the first PPG signal and the second PPG signal. The operations of 1435 may be performed in accordance with examples as disclosed herein. In some examples, aspects of the operations of 1435 may be performed by a PPG analysis component 1130 as described with reference to FIG. 11.

At 1440, the method may include determining one or more blood oxygen saturation metrics for the user during the time interval based at least in part on the comparison of the first PPG signal and the second PPG signal. The operations of 1440 may be performed in accordance with examples as disclosed herein. In some examples, aspects of the operations of 1440 may be performed by a blood oxygen component 1135 as described with reference to FIG. 11.

At 1445, the method may include causing a GUI to display an indication of the one or more blood oxygen saturation metrics. The operations of 1445 may be performed in accordance with examples as disclosed herein. In some examples, aspects of the operations of 1445 may be performed by a user interface component 1140 as described with reference to FIG. 11.

It should be noted that the methods described above describe possible implementations, and that the operations and the steps may be rearranged or otherwise modified and that other implementations are possible. Furthermore, aspects from two or more of the methods may be combined.

A method is described. The method may include receiving, via a wearable device, a first PPG signal for a user acquired during a time interval using a first set of PPG sensors, receiving, via the wearable device, a second PPG signal for the user acquired during the time interval using a second set of PPG sensors that are different from the first set of PPG sensors, comparing the first PPG signal and the second PPG signal, determining one or more blood oxygen saturation metrics for the user during the time interval based at least in part on the comparison of the first PPG signal and the second PPG signal, and causing a GUI to display an indication of the one or more blood oxygen saturation metrics.

An apparatus is described. The apparatus may include a processor, memory coupled with the processor, and instructions stored in the memory. The instructions may be executable by the processor to cause the apparatus to receive, via a wearable device, a first PPG signal for a user acquired during a time interval using a first set of PPG sensors, receive, via the wearable device, a second PPG signal for the user acquired during the time interval using a second set of PPG sensors that are different from the first set of PPG sensors, compare the first PPG signal and the second PPG signal, determine one or more blood oxygen saturation metrics for the user during the time interval based at least in part on the comparison of the first PPG signal and the second PPG signal, and cause a GUI to display an indication of the one or more blood oxygen saturation metrics.

Another apparatus is described. The apparatus may include means for receiving, via a wearable device, a first PPG signal for a user acquired during a time interval using a first set of PPG sensors, means for receiving, via the wearable device, a second PPG signal for the user acquired during the time interval using a second set of PPG sensors that are different from the first set of PPG sensors, means for comparing the first PPG signal and the second PPG signal, means for determining one or more blood oxygen saturation metrics for the user during the time interval based at least in part on the comparison of the first PPG signal and the second PPG signal, and means for causing a GUI to display an indication of the one or more blood oxygen saturation metrics.

A non-transitory computer-readable medium storing code is described. The code may include instructions executable by a processor to receive, via a wearable device, a first PPG signal for a user acquired during a time interval using a first set of PPG sensors, receive, via the wearable device, a second PPG signal for the user acquired during the time interval using a second set of PPG sensors that are different from the first set of PPG sensors, compare the first PPG signal and the second PPG signal, determine one or more blood oxygen saturation metrics for the user during the time interval based at least in part on the comparison of the first PPG signal and the second PPG signal, and cause a GUI to display an indication of the one or more blood oxygen saturation metrics.

In some examples of the method, apparatuses, and non-transitory computer-readable medium described herein, receiving the first PPG signal and the second PPG signal may include operations, features, means, or instructions for receiving a first PPG measurement utilizing a first channel between a first subset of the first set of PPG sensors, receiving a second PPG measurement utilizing a second channel between a second subset of the first set of PPG sensors, wherein the first PPG signal may be based at least in part on the first PPG measurement and the second PPG measurement, receiving a third PPG measurement utilizing a third channel between a first subset of the second set of PPG sensors, and receiving a fourth PPG measurement utilizing a fourth channel between a second subset of the second set of PPG sensors, wherein the second PPG signal may be based at least in part on the third PPG measurement and the fourth PPG measurement.

In some examples of the method, apparatuses, and non-transitory computer-readable medium described herein, the first set of PPG sensors comprises a first light source, a first photodetector, and a second photodetector, the first channel comprises a channel between the first light source and the first photodetector, the second channel comprises a channel between the first light source and the second photodetector, the second set of PPG sensors comprises a second light source, the first photodetector, and the second photodetector, the third channel comprises a channel between the second light source and the first photodetector, the fourth channel comprises a channel between the second light source and the second photodetector, and the second light source may be configured to generate light with a different wavelength as compared to light generated via the first light source.

In some examples of the method, apparatuses, and non-transitory computer-readable medium described herein, receiving the first PPG signal and the second PPG signal may include operations, features, means, or instructions for selectively controlling an activation state of the first light source and the second light source such that the first light source and the second light source may be simultaneously in an active activation state.

In some examples of the method, apparatuses, and non-transitory computer-readable medium described herein, receiving the first PPG signal and the second PPG signal may include operations, features, means, or instructions for sequentially controlling an activation state of the first light source and the second light source such that the first light source may be in an active activation state when the second light source may be in an inactive activation state, and vice versa.

Some examples of the method, apparatuses, and non-transitory computer-readable medium described herein may further include operations, features, means, or instructions for combining the first PPG measurement and the second PPG measurement to generate the first PPG signal and combining the third PPG measurement and the fourth PPG measurement to generate the second PPG signal.

In some examples of the method, apparatuses, and non-transitory computer-readable medium described herein, the first PPG measurement and the second PPG measurement and the third PPG measurement and the fourth PPG measurement, respectively, may be combined using one or more mathematical operations, the one or more mathematical operations comprising an averaging operation, a weighted averaging operation, or both.

Some examples of the method, apparatuses, and non-transitory computer-readable medium described herein may further include operations, features, means, or instructions for receiving a third PPG signal for the user acquired during the time interval using a third set of PPG sensors that may be different from the first set of PPG sensors and the second set of PPG sensors.

In some examples of the method, apparatuses, and non-transitory computer-readable medium described herein, receiving the third PPG signal may include operations, features, means, or instructions for receiving a fifth PPG measurement utilizing a fifth channel between a first subset of the third set of PPG sensors and receiving a sixth PPG measurement utilizing a sixth channel between a second subset of the third set of PPG sensors, wherein the third PPG signal may be based at least in part on the fifth PPG measurement and the sixth PPG measurement.

In some examples of the method, apparatuses, and non-transitory computer-readable medium described herein, the first set of PPG sensors, the second set of PPG sensors, and the third set of PPG sensors use three different wavelengths.

Some examples of the method, apparatuses, and non-transitory computer-readable medium described herein may further include operations, features, means, or instructions for determining a ratio between the first PPG signal and the second PPG signal, wherein determining the one or more blood oxygen saturation metrics may be based at least in part on the ratio of the first PPG signal and the second PPG signal.

Some examples of the method, apparatuses, and non-transitory computer-readable medium described herein may further include operations, features, means, or instructions for determining a first perfusion index between a first amplitude and a first baseline amplitude level of the first PPG signal and determining a second perfusion index between a second amplitude and a second baseline amplitude level of the second PPG signal, wherein determining the ratio of the first PPG signal and the second PPG signal comprises determining a ratio between the first perfusion index and the second perfusion index.

Some examples of the method, apparatuses, and non-transitory computer-readable medium described herein may further include operations, features, means, or instructions for generating a blood oxygen saturation signal based at least in part on the comparison of the first PPG signal and the second PPG signal and identifying one or more calibration coefficients for blood oxygen saturation calculations, wherein the one or more blood oxygen saturation metrics may be determined based at least in part on the blood oxygen saturation signal and the one or more calibration coefficients.

Some examples of the method, apparatuses, and non-transitory computer-readable medium described herein may further include operations, features, means, or instructions for acquiring temperature data during the time interval and modifying the first PPG signal and the second PPG signal based at least in part on the temperature data, wherein determining the one or more blood oxygen saturation metrics may be based at least in part on modifying the first PPG signal and the second PPG signal.

In some examples of the method, apparatuses, and non-transitory computer-readable medium described herein, the first set of PPG sensors comprises a first light source and a first set of one or more photodetectors, the second set of PPG sensors comprises a second light source and a second set of one or more photodetectors, and each of the first light source, the second light source, the first set of one or more photodetectors, and the second set of one or more photodetectors may be positioned at different radial positions relative to an axis of the wearable device and along an inner circumference of the wearable device.

Some examples of the method, apparatuses, and non-transitory computer-readable medium described herein may further include operations, features, means, or instructions for receiving physiological data associated with the user via the wearable device, the physiological data comprising at least motion data or temperature data, or both, wherein receiving the first PPG signal, the second PPG signal, or both, may be based at least in part on the motion data satisfying a threshold motion metric and the temperature data satisfying a threshold temperature metric.

Some examples of the method, apparatuses, and non-transitory computer-readable medium described herein may further include operations, features, means, or instructions for determining a breathing rate for the user based at least in part on the motion data, the first PPG signal, the second PPG signal, or a combination thereof and updating the one or more blood oxygen saturation metrics for the user based at least in part on determining the breathing rate for the user.

In some examples of the method, apparatuses, and non-transitory computer-readable medium described herein, the motion data satisfies the threshold motion metric if the motion data may be less than or equal to the threshold motion metric and the temperature data satisfies the threshold temperature metric if the temperature may be greater than or equal to the threshold temperature metric.

Some examples of the method, apparatuses, and non-transitory computer-readable medium described herein may further include operations, features, means, or instructions for receiving physiological data associated with the user via the wearable device, the physiological data comprising at least heart rate data or PPG signal feature data, or both, wherein receiving the first PPG signal, the second PPG signal, or both, may be based at least in part on the heart rate satisfying a threshold heart rate metric and the PPG signal feature data satisfying a threshold metric.

Some examples of the method, apparatuses, and non-transitory computer-readable medium described herein may further include operations, features, means, or instructions for determining a variation of the one or more blood oxygen saturation metrics for the user during the time interval and transmitting, to a user device, an indication of the one or more blood oxygen saturation metrics based at least in part on the variation exceeding a threshold, wherein displaying an indication of the one or more blood oxygen saturation metrics is based at least in part on the transmitting.

Some examples of the method, apparatuses, and non-transitory computer-readable medium described herein may further include operations, features, means, or instructions for causing the graphical user interface to display a sleep disturbance alert based at least in part on the variation of the one or more blood oxygen saturation metrics exceeding the threshold.

In some examples of the method, apparatuses, and non-transitory computer-readable medium described herein, at least one photodetector may be included within both the first set of PPG sensors and the second set of PPG sensors.

In some examples of the method, apparatuses, and non-transitory computer-readable medium described herein, the first PPG signal, the second PPG signal, or both, may be based at least in part on light that may be transmitted through tissue of the user and light that may be reflected by the tissue of the user.

In some examples of the method, apparatuses, and non-transitory computer-readable medium described herein, the first set of PPG sensors may be configured to acquire the first PPG signal using light of a first wavelength and the second set of PPG sensors may be configured to acquire the second PPG signal using light of a second wavelength that may be different from the first wavelength.

In some examples of the method, apparatuses, and non-transitory computer-readable medium described herein, the first set of PPG sensors comprise at least one red light-emitting diode and the second set of PPG sensors comprise at least one infrared light-emitting diode.

In some examples of the method, apparatuses, and non-transitory computer-readable medium described herein, the at least one red light-emitting diode emits light of 740-760 nm wavelength.

In some examples of the method, apparatuses, and non-transitory computer-readable medium described herein, the first set of PPG sensors comprise at least one laser diode and the second set of PPG sensors comprise at least one laser diode.

In some examples of the method, apparatuses, and non-transitory computer-readable medium described herein, the first set of PPG sensors may be angled towards a first set of one or more photodetectors and the second set of PPG sensors may be angled towards a second set of one or more photodetectors.

In some examples of the method, apparatuses, and non-transitory computer-readable medium described herein, the first PPG signal, the second PPG signal, or both, may be acquired from the user based on arterial blood flow.

In some examples of the method, apparatuses, and non-transitory computer-readable medium described herein, the wearable device comprises a wearable ring device.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for measuring blood oxygen saturation for a user comprising:
receiving, via a wearable device, a first photoplethysmogram (PPG) signal for a user acquired during a time interval using a first set of PPG sensors;
receiving, via the wearable device, a second PPG signal for the user acquired during the time interval using a second set of PPG sensors that are different from the first set of PPG sensors;
comparing the first PPG signal and the second PPG signal;
determining one or more blood oxygen saturation metrics for the user during the time interval based at least in part on the comparison of the first PPG signal and the second PPG signal; and
causing a graphical user interface to display an indication of the one or more blood oxygen saturation metrics.

2. The method of claim 1, wherein receiving the first PPG signal and the second PPG signal comprises:
receiving a first PPG measurement utilizing a first channel between a first subset of the first set of PPG sensors;
receiving a second PPG measurement utilizing a second channel between a second subset of the first set of PPG sensors, wherein the first PPG signal is based at least in part on the first PPG measurement and the second PPG measurement;
receiving a third PPG measurement utilizing a third channel between a first subset of the second set of PPG sensors; and
receiving a fourth PPG measurement utilizing a fourth channel between a second subset of the second set of PPG sensors, wherein the second PPG signal is based at least in part on the third PPG measurement and the fourth PPG measurement.

3. The method of claim 2, wherein the first set of PPG sensors comprises a first light source, a first photodetector, and a second photodetector, wherein the first channel comprises a channel between the first light source and the first photodetector, and wherein the second channel comprises a channel between the first light source and the second photodetector, and wherein the second set of PPG sensors comprises a second light source, the first photodetector, and the second photodetector, wherein the third channel comprises a channel between the second light source and the first photodetector, and wherein the fourth channel comprises a channel between the second light source and the second photodetector, wherein the second light source is configured to generate light with a different wavelength as compared to light generated via the first light source.

4. The method of claim 3, wherein receiving the first PPG signal and the second PPG signal comprises:
selectively controlling an activation state of the first light source and the second light source such that the first light source and the second light source are simultaneously in an active activation state.

5. The method of claim 3, wherein receiving the first PPG signal and the second PPG signal comprises:
sequentially controlling an activation state of the first light source and the second light source such that the first light source is in an active activation state when the second light source is in an inactive activation state, and vice versa.

6. The method of claim 2, further comprising:
combining the first PPG measurement and the second PPG measurement to generate the first PPG signal; and
combining the third PPG measurement and the fourth PPG measurement to generate the second PPG signal.

7. The method of claim 6, wherein the first PPG measurement and the second PPG measurements and the third PPG measurement and the fourth PPG measurement, respectively, are combined using one or more mathematical operations, the one or more mathematical operations comprising an averaging operation, a weighted averaging operation, or both.

8. The method of claim 2, further comprising:
receiving a third PPG signal for the user acquired during the time interval using a third set of PPG sensors that are different from the first set of PPG sensors and the second set of PPG sensors.

9. The method of claim 8, wherein receiving the third PPG signal comprises:
receiving a fifth PPG measurement utilizing a fifth channel between a first subset of the third set of PPG sensors; and
receiving a sixth PPG measurement utilizing a sixth channel between a second subset of the third set of PPG sensors, wherein the third PPG signal is based at least in part on the fifth PPG measurement and the sixth PPG measurement.

10. The method of claim 8, wherein the first set of PPG sensors, the second set of PPG sensors, and the third set of PPG sensors use three different wavelengths.

11. The method of claim 1, further comprising:
determining a ratio between the first PPG signal and the second PPG signal, wherein determining the one or more blood oxygen saturation metrics is based at least in part on the ratio of the first PPG signal and the second PPG signal.

12. The method of claim 11, further comprising:
determining a first perfusion index between a first amplitude and a first baseline amplitude level of the first PPG signal; and
determining a second perfusion index between a second amplitude and a second baseline amplitude level of the second PPG signal, wherein determining the ratio of the first PPG signal and the second PPG signal comprises determining a ratio between the first perfusion index and the second perfusion index.

13. The method of claim 1, further comprising:
generating a blood oxygen saturation signal based at least in part on the comparison of the first PPG signal and the second PPG signal; and
identifying one or more calibration coefficients for blood oxygen saturation calculations, wherein the one or more blood oxygen saturation metrics are determined based at least in part on the blood oxygen saturation signal and the one or more calibration coefficients.

14. The method of claim 13, further comprising:
acquiring temperature data during the time interval; and
modifying the first PPG signal and the second PPG signal based at least in part on the temperature data, wherein determining the one or more blood oxygen saturation metrics is based at least in part on modifying the first PPG signal and the second PPG signal.

15. The method of claim 1, wherein the first set of PPG sensors comprises a first light source and a first set of one or more photodetectors, and wherein the second set of PPG sensors comprises a second light source and a second set of one or more photodetectors, wherein each of the first light source, the second light source, the first set of one or more photodetectors, and the second set of one or more photodetectors are positioned at different radial positions relative to an axis of the wearable device and along an inner circumference of the wearable device.

16. The method of claim 1, further comprising:
receiving physiological data associated with the user via the wearable device, the physiological data comprising at least motion data or temperature data, or both, wherein receiving the first PPG signal, the second PPG signal, or both, is based at least in part on the motion data satisfying a threshold motion metric and the temperature data satisfying a threshold temperature metric.

17. The method of claim 16, further comprising:
determining a breathing rate for the user based at least in part on the motion data, the first PPG signal, the second PPG signal, or a combination thereof; and
updating the one or more blood oxygen saturation metrics for the user based at least in part on determining the breathing rate for the user.

18. The method of claim 16, wherein the motion data satisfies the threshold motion metric if the motion data is less than or equal to the threshold motion metric, and wherein the temperature data satisfies the threshold temperature metric if the temperature is greater than or equal to the threshold temperature metric.

19. The method of claim 1, further comprising:
receiving physiological data associated with the user via the wearable device, the physiological data comprising at least heart rate data or PPG signal feature data, or both, wherein receiving the first PPG signal, the second PPG signal, or both, is based at least in part on the heart rate satisfying a threshold heart rate metric and the PPG signal feature data satisfying a threshold PPG signal feature metric.

20. The method of claim 1, further comprising:
determining a variation of the one or more blood oxygen saturation metrics for the user during the time interval;
transmitting, to a user device, an indication of the one or more blood oxygen saturation metrics based at least in part on the variation exceeding a threshold, wherein displaying an indication of the one or more blood oxygen saturation metrics is based at least in part on the transmitting; and
causing the graphical user interface to display a sleep disturbance alert based at least in part on the variation of the one or more blood oxygen saturation metrics exceeding the threshold.
